SEQUENCE LISTING (3 sequences)

SEQ ID NO: 1 - DNA, 19 bp, Artificial Sequence
  Description of Artificial Sequence Synthetic oligonucleotide
gcccttcactactgttgac

SEQ ID NO: 2 - DNA, 19 bp, Artificial Sequence
  Description of Artificial Sequence Synthetic oligonucleotide
acgctgagtacttcgaaat

SEQ ID NO: 3 - DNA, 3012 bp, Homo sapiens
gtaatttattttacttctgtgtcctaagggtaatttctcaggattgttttcaaattgcttttttaggggaaataggtcatttgctatattacaagcaatccccaaattttatggtcttccaggaaaagttattaccgtttatgatactaacagttcctgagacttagctatgatcagtatgttcatgaggtggagcagttcctgtgttgcagcttttaacaacagatggcattcattaaatcacaaagtatgttaaaggtcacaaaagcaaaataactgtctgaggctaaggcccacgtgggacagtctaatacccatgagtactcaacttgccttgatgtctgagctttccagtgcaatgtgaatttgagcagccagaaatctattagtagaaagcaagacagattaatataggttaaaacaatgatttaaatatgtttctcccaataattatctctttccctggaatcaacttgtatgaaaccttgtcaaaatgtactccacaagtatgtacaattaagtattttaaaaataaatggcaaacattaaaaacaagagtgaatactcaagtagatttgtcatgggatttttataagaagactggtatcaggtaatgtatctttaaagactaggctgctctgctgacgtagtaaccattttttattcctttactttcctaatagccttgctttcacttaagaaaaaaaaaggctaggcacggtggctcacgcctgtaatcccagcactttgggaggccaaggtgagtagatcacctgaggtcaggagttcgagacaagcctggccaacgtggagaaaacctgtctctactaaaagtacaaaaattagccaggcatggtggtgggcacctgtaatcccagctactcaggaggttgaggcaggagaattgcttgaacccaagaggtggaagttgcagtgagctgagatcatgccactgccctccagcttgggcggcaacaacaacaacaaaaaaagattcccaggcttcaccctctacagtctatggaaagtcatgggagtctattaaaaaaaaacagacactataaaccaaattaaaagatgggaaaatttttctatcacgttttaatatgtgatatccaaactcccattaagaatttttatatcagtaataagataaaactcagaagaaaaatgcccaattaacaagaagagcttcacctcttaaataatcagaaaaagaaaagcacataatatttacgtcaaagtatcaaaaatgtaaaagttttataataggcattagcaaggcagacggatacaggcacacatctaactctatgtaggatttaacttggaagacaatttgcatttcaatcaatcaaaaaccatttttttttttgagatgatctccctgtgttgcccagactggcctcaagctcctgggcttgagcagtcctacagtctgagcctccaaaagtactggcaaaacaggcgtgattgaccatgcccagcctgtatataaggatattcatttgcagcattgtttgcaacacaaaaatgtaaaaccaaccggtgtttaatatgcaagatgttaatttatggtacattcttgctgtggaaagctatgcatctgttagaggtgggtctatatgttctgatacaggcagaactctaagagctattaagtgaagaaaaggctgaaaatacatatggcataattccatatatgttaaattgttctatattttaaatgttatacacgtttccatttgtatttacgtaataataatgatccctgagctgtactgttgtatacatgctacaagaaagacccatttaatccccacagcctatgatgaattatccacattctacaggtgacaaaatagaggcacaaagttaagtaatttttgtcaggtgagatttaaacccaggcattctgactcctgtataaccattaagatatgcagagaaagaaaactggaaagatacatattgctgaagatacttattataggaagaggaggggggagggtgaaggaatttgcaagtttttcatagatgtttccatattgtttgaatctcttacaaaatatgttcagcatatttttaaagagaaaatttggggcaaaatacttatttttgtattatgtaaacaaattttaaaataatgtgtggctgggtgcgctggctcacacctgtaatcccaacactttaggaggctgaggcaagaggattgcttgagcccaggagttcaagaccagcctgggtgacatggcaaaactccatctctactaaaaatacaaaaaattagccagtcgtggtggcgcacacctatggtcccacctacccaggatgctgagatgggaggatcacttgagcccaggaagtcaaggctgcaggaagctgtgatcgcaccactgcactcccacctgggcaacagagtgagacccggtcaccaaaaaacaaaaaaaacaaaaaaaattggtaatcgttttcttcagacattttccgggttcctctgcttaacttgtataggaagtctgaggtttttgtgttggtctttaccttttttttttttttttttttttaagatggagtctcattctgttgcccaggctggagtgcagtggcatgatcttggctcctgcaacctccgcctcctgggttcaagtgattctcctgcctcagcctcctgagtagccgggactacaggcgcatgccacgatgcctggctaattttttgtatttttagtagagatggggtttcaccatgttagctaggacggtctcgatctcctgacctcgtgatccgcccacctcggcctcccaaagtgctggaattacaggtgtgagccaccacgcccggccctgatctttacatttttaaatattgcattagtgaaccgtgtactgattttgtgatcatagataacccagttaaatattaagtcttaattatcacttagtattttacaacctcagttgcagttataaagtaagggttccacatacctcctaacagttcctagaaaa

(19) United States
(12) Patent Application Publication
(10) Pub. No.: US 2019/0046490 A1
(43) Pub. Date: Feb. 14, 2019

(54) METHODS OF TREATING LACTOSE INTOLERANCE

(71) Applicant: Nogra Pharma Limited, Dublin 2 (IE)

(72) Inventors: Marie McNulty, Dublin (IE); Francesca Viti, Salorino (CH); Salvatore Bellinvia, Mendrisio (CH)

(21) Appl. No.: 16/078,555

(22) PCT Filed: Feb. 27, 2017
(86) PCT No.: PCT/EP2017/054526

§ 371 Date: Aug. 21, 2018

Related U.S. Application Data
(60) Provisional application No. 62/300,376, filed on Feb. 26, 2016.

(51) Int. Cl. A61K 31/201; A61P 1/00

(52) U.S. Cl. CPC A61K 31/201; A61P 1/00

(57) ABSTRACT

Disclosed herein in part are methods for treating lactose intolerance, including administering fatty acid compounds that modulate PPARγ receptors.

### CROSS REFERENCE TO RELATED APPLICATION

This application claims priority to and the benefit of U.S. Provisional Patent Application No. 62/300,376, filed Feb. 26, 2016, the entire contents of which are herein incorporated by reference.

### BACKGROUND

Lactase protein is a disaccharidase (β-galactosidase) expressed on the tips of the villi of the small intestine having the ability to hydrolyze lactose into galactose and glucose. Inadequate lactase-phlorizin hydrolase (LPH) activity is responsible for lactose intolerance/malabsorption leading to diarrhea, abdominal pain or bloating after lactose ingestion. Primary lactase deficiency (or lactase non persistence or hypolactasia) is the main cause of lactose intolerance, due to the relative or absolute absence of lactase expression in the small bowel, occurring in childhood at various ages and in different racial groups. Approximately 70% of the world's population has primary lactase deficiency. The percentage of lactose deficiency varies according to ethnicity and is related to the use of dairy products in the diet reaching up to 20% of North European, 40% of Mediterranean European, 80% of Africans, and 90% of Asian population. No curative treatments for primary lactose intolerance are currently available, with typical treatments for lactose intolerance including lactose exclusion (leading to nutritional impairment) or expansive regimen such as the use of lactose deficient milk or lactase supplementation. In the United States alone, the annual financial burden of lactose intolerance is estimated to be nearly 2 billion dollars.

It has been reported that two particular single nucleotide polymorphisms (SNP) are tightly associated with adult-type hypolactasia. A C at position 13910 (C₁₃₉₁₀) upstream of the lactase gene is 100% associated and a G at position 22018 (G₂₂₀₁₈) is more than 95% associated with lactase non-persistence in the Finnish population. Expression of LPH mRNA in the intestinal mucosa in individuals with T₁₃₉₁₀ and A₂₂₀₁₈ is higher than found in individuals with C₁₃₉₁₀ and G₂₂₀₁₈, suggesting transcriptional regulation of the LPH gene. However, much of the regulation of the LPH gene remains unknown. In particular, although several elements of the genetics of hypolactasia have been elucidated, no modulator able to increase LCT expression has yet been identified. Accordingly, effective agents that are useful in the treatment of lactose intolerance and related disorders are needed.

Peroxisome Proliferator Activated Receptors (PPARs) are members of the nuclear hormone receptor super family, which are ligand-activated transcription factors regulating gene expression. PPARs play a role in the regulation of cell differentiation, development and metabolism of higher organisms.

Three types of PPAR have been identified: alpha, expressed in the liver, kidney, heart and other tissues and organs, beta/delta expressed for example in the brain, and gamma, expressed in three forms: gamma1, gamma2, and gamma3. PPARγ has been associated with stimulation of keratinocyte differentiation and is a master gene for the control of glucose homeostasis and lipid metabolism. As such, PPARγ has served as a drug target for a number of disease states including skin disorders such as psoriasis and atopic dermatitis type 2 diabetes with the development of the thiazolidinedione (TZD) class of drugs. To date, most studies have evaluated the role of PPARγ in major metabolic organs such as the liver, adipocytes, pancreas or skeletal muscles. Intestinal epithelial cells (IEC) constitute another major source of PPARγ, however, the role of PPARγ in IEC during carbohydrate metabolism has been poorly investigated.

### SUMMARY

Described herein are methods for treating and/or ameliorating lactose intolerance or lactase deficiency in a patient in need thereof, the method comprising administering a composition comprising an isolated fatty acid to the patient. Also described herein are methods for stimulating lactase gene expression in a patient in need thereof, comprising administering a composition comprising an isolated fatty acid to said patient, and methods for treating diarrhea, abdominal pain and/or bloating after lactose ingestion in a lactose intolerant patient in need thereof, comprising administering a composition comprising an isolated fatty acid. In some aspects the disclosure is directed to a method for treating and/or ameliorating lactose intolerance or lactase deficiency in a patient in need thereof, where the method includes administering to the patient a composition consisting essentially of a fatty acid, for example, a conjugated linoleic acid. In some embodiments, a fatty acid is a naturally occurring fatty acid, for example, a naturally occurring conjugated linoleic acid.

In certain embodiments, the administering may be before, after, or substantially concurrent with the consumption of a food that includes a dairy product. In some embodiments, the methods include administering a composition that includes a fatty acid daily, weekly, or as needed over a time period of 3 months, 6 months, 1 year, or more. A patient (e.g., a human patient) may also be suffering from one or more of: gastroenteritis, celiac disease, Crohn's disease, and/or bacterial overgrowth, and/or undergoing radiation therapy and/or chemotherapy. In certain embodiments, the fatty acid is a conjugated linoleic acid, e.g., trans-10, cis-12 conjugated linoleic acid isomer, cis-9, trans-11 conjugated linoleic acid isomer, or a mixture thereof.

In other aspects, a food product that includes a therapeutically effective amount of a fatty acid to ameliorate lactose intolerance in a patient is provided. In some embodiments, the food product includes a therapeutically effective amount of a fatty acid to ameliorate lactose intolerance in a patient and, optionally, a dairy component, e.g., whey, milk, cheese or cream. In certain embodiments, the fatty acid is a conjugated linoleic acid, e.g. the trans-10, cis-12 conjugated linoleic acid isomer, the cis-9, trans-11 conjugated linoleic acid isomer, or a mixture thereof.

Also provided herein is a food product comprising a fatty acid, for example, a conjugated linoleic acid, in an amount significantly greater than a naturally occurring amount of a fatty acid, for example, a naturally occurring amount of a conjugated linoleic acid, in the food product, e.g., wherein the amount of the fatty acid (for example, a conjugated linoleic acid) is about 5%, about 10%, about 50%, about 100%, or more than about 100% by weight greater than a naturally occurring amount of the fatty acid (for example, a naturally occurring amount of a conjugated linoleic acid) in the food product. In some embodiments, the food product includes a conjugated linoleic acid where the conjugated linoleic acid is a trans-10, cis-12 conjugated linoleic acid isomer, a cis-9, trans-11 conjugated linoleic acid isomer, or a mixture thereof.

In another aspect, the disclosure is directed to nutraceutical compositions that include a therapeutically effective amount of a fatty acid, for example, a conjugated linoleic acid, where the therapeutically effective amount of the fatty acid, for example, the therapeutically effective amount of a conjugated linoleic acid, substantially prevents, ameliorates, or treats lactose intolerance in a human patient when orally administered or consumed by the patient. In some embodiments, a nutraceutical composition includes a conjugated linoleic acid, where the conjugated linoleic acid is a trans-10, cis-12 conjugated linoleic acid isomer, a cis-9, trans-11 conjugated linoleic acid isomer, or a mixture thereof.

In yet another aspect, the disclosure is directed to pharmaceutical formulation for oral administration of a fatty acid. In some embodiments, a pharmaceutical formulation of the disclosure includes a fatty acid, a pharmaceutically acceptable filler, and an enteric coating. In some embodiments, a pharmaceutical formulation includes a fatty acid that is a conjugated linoleic acid. In some embodiments, a pharmaceutical formulation includes a fatty acid where the fatty acid is a trans-10, cis-12 conjugated linoleic acid isomer, a cis-9, trans-11 conjugated linoleic acid isomer, or a mixture thereof. In some embodiments, a pharmaceutical formulation of the disclosure includes a disintegrant. In some embodiments, a pharmaceutical formulation of the disclosure includes a lubricant. In some embodiments, a pharmaceutical formulation of the disclosure includes an enteric coating, where the enteric coating is about 1% to about 10%, about 5% to about 10%, about 8% to about 10%, about 8% to about 12%, about 8% to about 15%, about 8% to about 20%, about 10% to about 12%, about 10% to about 18%, or about 15% to about 20% by weight of the pharmaceutical formulation. In some embodiments of a disclosed pharmaceutical formulation, the enteric coating is ethylacrylate methacrylic acid.

In some embodiments, a pharmaceutical formulation of the disclosure, when orally administered to a patient, results in delivering the fatty acid to the duodenum of the patient and/or the jejunum of the patient. In some embodiments, a pharmaceutical formulation of the disclosure, when orally administered to a patient, results in release of fatty acid at a pH value of about 4.5, about 5, about 5.5, about 6, about 6.5, or about 7. In some embodiments, a pharmaceutical formulation of the disclosure, when administered to a patient, results in release of fatty acid in the gastrointestinal tract in an environment of about pH 4.5, about pH 5, about pH 5.5, about pH 6, about pH 6.5, or about pH 7.

In some embodiments, a pharmaceutical formulation of the disclosure, when orally administered to a patient results in amelioration or treatment of lactose intolerance or lactase deficiency in the patient. In some embodiments, a pharmaceutical formulation of the disclosure results in amelioration or treatment of lactose intolerance or lactase deficiency in the patient after the formulation is administered a defined number of times over a defined period of time, for example, after 1 time, after 2 times, after 3 times, after 4 times, after 5 times, after 6 times, after 7 times, after 8 times, after 9 times, after 10 times, or after more than 10 times over the course of 1 hour, 1 day, 1 week, or 1 month.

In some aspects, the disclosure is directed to a fatty acid, for example, linoleic acid, for example, conjugated linoleic acid, for use as a medicament, for example, for treating, preventing, managing, and/or ameliorating lactose intolerance or lactase deficiency in a patient in need thereof. In some aspects, the disclosure is directed to a fatty acid for use in treating, preventing, managing, and/or ameliorating lactose intolerance or lactase deficiency in a patient in need thereof. In some embodiments, the fatty acid for use in treating, preventing, managing, and/or ameliorating lactose intolerance or lactase deficiency in a patient in need thereof is for use in any of the methods disclosed herein. Use of a fatty acid, for example, linoleic acid, for example, conjugated linoleic acid, in the manufacture of a medicament for the treatment, prevention, management, and/or amelioration of lactose intolerance or lactase deficiency by a method described herein is also provided herein.

### BRIEF DESCRIPTION OF THE FIGURES

FIG. 1A depicts quantitative PCR (qPCR) analysis showing induction of LCT mRNA expression by 1 mM 3-(4′-aminophenyl)2-methoxypropionic acid (GED) in Caco-2 cells relative to unstimulated (CTL) cells (CTL v. 1 mM GED, p<0.0001). FIG. 1B depicts qPCR analysis showing induction of LCT mRNA expression by 1 μM pioglitazone (Pio) in Caco-2 cells relative to CTL cells (CTL v. 1 μM Pio, p<0.0001). Results in FIGS. 1A and 1B represent the mean±standard error of the mean (SEM) of 4 independent experiments. The fold change of LCT gene expression is normalized to GAPDH mRNA expression levels. FIG. 1C depicts qPCR analysis showing induction of LCT mRNA expression by 30 GED in Caco-2 cells relative to CTL cells (CTL v. 30 mM GED, p=0.0002). FIG. 1D depicts qPCR analysis showing induction of LCT mRNA expression by 30 5-aminosalicylic acid (5-ASA) in Caco-2 cells relative to CTL cells (CTL v. 30 mM 5-ASA, p=0.0002). The expression level measured in control cells (arbitrarily defined as one) was used as a reference in each of FIGS. 1A-1D.

FIG. 2A depicts the dose-effect of GED on LCT mRNA expression in Caco-2 cells, where cells were stimulated with 0.1 mM, 1 mM, or 30 mM GED, and LCT mRNA expression relative to controls (CTRL; DMEM) was determined by qRT-PCR. FIG. 2B depicts the dose-effect of Pio on LCT mRNA expression in Caco-2 cells, where cells were stimulated with 0.1 μM, 1 μM, or 10 μM Pio, and LCT mRNA expression relative to controls (CTRL; DMSO) was determined by qRT-PCR. Results in FIGS. 2A and 2B represent the mean±SEM of 2 to 3 independent experiments performed in triplicate (*, P<0.05; *** P<0.001; NS, not significant). The expression level measured in CTRL cells was used as a reference.

FIG. 3 is a bar graph display of LCT protein expression assessed by immunoprecipitation assay. LCT protein was immunoprecipitated from Caco-2 cells either stimulated with 1 mM GED (GED) or left unstimulated (CTRL). Bars represent LCT protein signal intensity relative to β-actin signal intensity. CTRL signal was arbitrarily defined as 100%.

FIG. 4A is a bar graph depicting LCT activity in Caco-2 cells after stimulation with 1mM GED (GED) or no stimulation (CTRL). Results represent the mean±SEM (3 independent experiments performed in triplicate) of the percentage of LCT activity compared to the activity in CTRL cells, arbitrarily defined as 100%.

FIG. 4B is a bar graph depicting LCT activity in Caco-2 cells after stimulation with 1 μM Pio (Pio) or no stimulation (CTRL). Results represent the mean±SEM (3 independent experiments in triplicate) of the percentage of LCT activity compared to the activity in CTRL cells, arbitrarily defined as 100%.

FIG. 4C is a bar graph depicting LCT activity in Caco-2 cells after stimulation with 1 mM GED (GED1mM), 30 mM GED (GED30 mM), 30 mM 5-ASA (5ASA30 mM), or no stimulation (CTRL). Lactase activity was significantly upregulated compared to CTRL samples following stimulation with 1 mM GED (CTRL v. GED 1 mM, p<0.005) and 30 mM GED (CTRL v. GED 30 mM, p<0.005).

FIG. 5 depicts the glucose uptake capacity of Caco-2 cells after 1 mM GED (GED 1 mM) and 1 μM pioglitazone (Pio 1 μM) stimulation or no stimulation (CTRL). The result is expressed in the amount of phosphorylation of the glucose analog 2-deoxyglucose (2-DG6P) measured in the cells (pmol). NS, not significant.

FIG. 6A depicts the relative expression level of sucrase-isomaltase (SIM) and maltase-glucoamylase (MGAM) mRNA compared to LCT mRNA in Caco-2 cells following stimulation with a PPARγ agonist as determined by qPCR.

FIG. 6B depicts the relative expression level of SIM mRNA in Caco-2 cells as determined by qPCR following stimulation with 1 mM GED (left) or 1 μM Pio (right) or left unstimulated (CTRL and DMSO). Results represent the mean±SEM (2 independent experiments performed in sextuplicate) of the fold change of expression of SIM mRNA normalized to GAPDH level. The expression level measured in control cells (arbitrarily defined as one) was used as reference. ** P<0.01; ***P<0.001; NS, not significant.

FIG. 6C depicts the relative expression level of MGAM mRNA in Caco-2 cells as determined by qPCR following stimulation with 1 mM GED (left) or 1 μM Pio (right) or left unstimulated (CTRL and DMSO). Results represent the mean±SEM (2 independent experiments performed in sextuplicate) of the fold change of expression of MGAM mRNA normalized to GAPDH level. The expression level measured in control cells (arbitrarily defined as one) was used as reference. ** P<0.01; NS, not significant.

FIG. 7 depicts the correlation of gene expression data as determined by microarray and qPCR analyses (r=0.754, p=0.0046).

FIG. 8 is a schematic of the PPAR response element (PPRE) identified by in silico analysis in the promoter region of the human LCT gene (up to 3,000 bp upstream of the putative transcription start site) and the direct repeat 1 (DR1) and direct repeat 2 (DR2) response elements located in the region. 8a and 8b denote the primer pair used to amplify the genomic region encompassing the DR2 located between nucleotides −223 to −210.

FIG. 9 depicts the nucleotide sequence of the PPRE (DR1 and DR2) in the human LCT promoter gene (up to 3,000 bp upstream to the transcription start point). The putative DR1's and DR2's identified in the 3,000 bp sequence of the LCT gene promoter are underlined. The underlined nucleotide sequence “TAAATA” denotes a potential TATA box. FIG. 9 discloses SEQ ID NO: 3.

FIG. 10 depicts a bar graph showing qPCR amplification signal of the 8a-8b fragment in a ChIP assay from Caco-2 cells either treated with GED (GED) or not treated (CTRL). Results are expressed as fold enrichment relative to CTRL cells.

FIG. 11 depicts results of a luciferase gene reporter assay in Caco-2 cells transfected with a reporter construct containing the DR2 response element upstream of a luciferase gene sequence (pGL4Luc PromLCT construct) or a control construct containing a luciferase gene sequence but no upstream DR2 sequence (pGL4Luc). Results represent the mean±SEM of luciferase activity normalized for protein content (2 independent experiments in triplicate) following stimulation with GED or no stimulation (CTL).

FIG. 12 depicts LCT mRNA expression as measured by qPCR (left) and LCT activity (right) in stably transfected PPARγ knock-down Caco-2 cells (ShPPAR) compared to stably transfected control cells (ShLuc). Results represent the mean±SEM of 3 independent experiments performed in triplicate or sextuplicate (**, P<0.01; ***, P<0.001).

FIG. 13 is a bar graph depicting the effect of the PPARγ antagonist GW9662 on GED-dependent induction of LCT mRNA expression in Caco-2 cells. LCT mRNA expression was determined by qPCR. Cells were treated with GW9662 (+GW9662) or left untreated (−GW9662) and then treated with GED (GED) or left untreated (Control). Results represent the mean±SEM (of 2 independent experiments performed in triplicate and sextuplicate) of the fold change in LCT mRNA expression, relative to cells that were not treated with GW9662 or GED (**, P<0.01; ***, P<0.001).

FIG. 14 is a bar graph depicting relative LCT mRNA expression levels in the proximal small intestine of control mice (CTRL) and mice that lack expression of PPARγ in intestinal epithelial cells (PPARγ^{ΔIEC}). Results represent the mean±standard deviation of the mean (SD; n=5; *, P<0.05).

FIG. 15A is a pair of graphs depicting relative expression of LCT mRNA (left) and PPARγ mRNA (right) in different sections of the gut of “not weaned” and “weaned” rats, as determined by qPCR. Results represent the mean±SD of the relative mRNA expression levels normalized to GAPDH levels (for each group n=6).

FIG. 15B is a graph depicting a correlation of LCT mRNA and PPARγ mRNA levels in the jejunum of weaned (squares) and not weaned (circles) rats.

FIG. 15C is a graph depicting a correlation of LCT mRNA and PPARγ mRNA levels in the duodenum of weaned (squares) and not weaned (circles) rats.

FIG. 16 is a bar graph depicting upregulation of lactase mRNA expression by epithelial cells in short term cultures of human duodenal biopsies following stimulation with the PPARγ modulator GED for 3 hours (GED 3H), 6 hours (GED 6H), or 10 hours (GED 10H) relative to unstimulated controls (CTL; CTL v. GED 6H, p=0.008).

FIG. 17 is a series of bar graphs depicting LCT mRNA expression measured by qPCR (left) and LCT activity (right) in Caco-2 cells stimulated with fenofibrate compared to unstimulated control cells (DMSO). Results represent the mean±SEM of 3 independent experiments performed in triplicate (NS, not significant).

FIG. 18A is a series of graphs depicting LCT mRNA expression (left) and LCT activity (right) measured in vivo in the proximal small intestine of C57BL/6 mice (top row) and Sprague-Dawley rats (bottom row) not treated (Control) or treated with 30 mg/g oral GED (GED) for 7 days. Results represent the sum of three independent experiments (mice, n=25-30; rats, n=21-23). Horizontal bars represent mean values (**, P<0.01; ***, P<0.001).

FIG. 18B is a graph depicting stool consistency score at different time points (DAY 0-4) in rats fed a control diet (CTRL diet) or a lactose-enriched diet (15% Lactose diet) and treated with GED (+GED) or not treated with GED (+CMC). Results represent the sum of two independent experiments (n=20 for each group; **, P<0.01; ***, P<0.001).

FIG. 18C is a graph depicting total short-chain fatty acids (SCFA) concentration (mmol/L) in the caecal contents of rats fed a control diet (Control diet) or a lactose-enriched diet (Lactose diet) and treated with GED (GED) or not treated with GED (CMC) for 4 days. Horizontal bars represent mean values (n=10 for each group; *, P<0.05; ***, P<0.001.

FIG. 19A depicts the chemical structures of a series of naturally occurring PPARγ ligands.

FIG. 19B is a bar graph depicting induction of LCT mRNA expression in Caco-2 cells as measured by qPCR in unstimulated cells (CTL) or following stimulation with 1 mM GED (GED 1 mM) or conjugated linoleic acid (CLA) at various concentrations (50 μM, 100 μM, 250 μM, 500 μM, 1000 μM). Results represent the mean±SEM of the fold change of LCT mRNA expression normalized to GAPDH mRNA levels, relative to LCT mRNA expression in CTL (n=3 independent experiments performed in quadruplicate; ***, P<0.001).

FIG. 19C is a bar graph depicting induction of LCT activity in Caco-2 cells in unstimulated cells (CTL) or following stimulation with 1 mM GED (GED 1 mM) or CLA at various concentrations (50 μM, 100 μM, 250 μM, 500 μM, 1000 μM). Results represent the mean±SEM of the fold change of LCT activity, relative to LCT activity in CTL (n=3 independent experiments performed in quadruplicate; *, P<0.05; ***, P<0.001).

FIG. 20A is a bar graph depicting LCT mRNA expression as measured by qPCR in stably transfected PPARγ knock-down Caco-2 cells (ShPPAR) and stably transfected control cells (ShLuc) not stimulated (CTRL) or stimulated with CLA at various concentrations (250 μM, 500 μM, 1000 μM). Results represent the mean±SEM of the fold change of LCT mRNA expression relative to LCT mRNA expression in CTRL (n=3 independent experiments performed in quadruplicate; **, P<0.01; ***, P<0.001; NS, not significant).

FIG. 20B is a bar graph depicting LCT mRNA activity in stably transfected PPARγ knock-down Caco-2 cells (ShPPAR) and stably transfected control cells (ShLuc) not stimulated (CTRL) or stimulated with 1 mM GED (GED 1 mM) or CLA at various concentrations (250 μM, 500 μM, 1000 μM). Results represent the mean±SEM of the fold change in LCT activity relative to LCT activity in CTRL (n=2 independent experiments performed in quadruplicate; *P<0.05; **, P<0.01; NS, not significant).

FIG. 21A is a schematic of an experimental design for analyzing the effects of feeding Sprague-Dawley rats a control diet supplemented with 0.5% carboxymethyl cellulose (Control diet+CMC; n=24 animals from 3 independent experiments) or a diet supplemented with 200 mg/kg/day CLA (Control diet+CLA 200 mg/Kg/day (Oral gavage); n=10 animals) or 30 mg/kg/day GED (Control diet+GED/Kg/day (Oral gavage); n=15 animals from 2 independent experiments) for 5 days (D₅).

FIG. 21B is a graph depicting individual data points and mean values (horizontal bars) for LCT mRNA expression levels (normalized to GAPDH mRNA expression) in duodenal tissue of rats fed a control diet supplemented with 0.5% carboxymethyl cellulose (CMC), 30 mg/kg/day GED (GED), or 200 mg/kg/day CLA (CLA), relative to CMC controls.

FIG. 21C is a graph depicting individual data points and mean values (horizontal bars) for LCT mRNA expression levels (normalized to GAPDH mRNA expression) in jejunal tissue of rats fed a control diet supplemented with 0.5% carboxymethyl cellulose (CMC), 30 mg/kg/day GED (GED), or 200 mg/kg/day CLA (CLA), relative to CMC controls.

FIG. 21D is a graph depicting individual data points and mean values (horizontal bars) for PPARγ mRNA expression levels (normalized to GAPDH mRNA expression) in duodenal tissue of rats fed a control diet supplemented with 0.5% carboxymethyl cellulose (CMC) or 200 mg/kg/day CLA (CLA), relative to CMC controls.

FIG. 21E is a graph depicting individual data points and mean values (horizontal bars) for PPARγ mRNA expression levels (normalized to GAPDH mRNA expression) in jejunal tissue of rats fed a control diet supplemented with 0.5% carboxymethyl cellulose (CMC) or 200 mg/kg/day CLA (CLA), relative to CMC controls.

FIG. 22A is a graph depicting the correlation between PPARγ (PPARg) and LCT (LCT) mRNA expression levels in the duodenal tissue of rats fed a control diet supplemented with 0.5% carboxymethyl cellulose.

FIG. 22B is a graph depicting the correlation between PPARγ (PPARg) and LCT (LCT) mRNA expression levels in the duodenal tissue of rats fed a control diet supplemented with 200 mg/kg/day CLA.

FIG. 22C is a graph depicting a correlative analysis of PPARγ (PPARg) and LCT (LCT) mRNA expression levels in the jejunal tissue of rats fed a control diet supplemented with 0.5% carboxymethyl cellulose.

FIG. 22D is a graph depicting a correlative analysis of PPARγ (PPARg) and LCT (LCT) mRNA expression levels in the jejunal tissue of rats fed a control diet supplemented with 0.5% carboxymethyl cellulose.

FIG. 22E is a graph depicting individual data points and mean values (horizontal bars) of fold change in LCT activity in duodenal tissue of rats fed a control diet supplemented with 0.5% carboxymethyl cellulose (CMC), 30 mg/kg/day GED (GED), or 200 mg/kg/day CLA (CLA) for 5 days.

FIG. 22F is a graph depicting individual data points and mean values (horizontal bars) of fold change in LCT activity in jejunal tissue of rats fed a control diet supplemented with 0.5% carboxymethyl cellulose (CMC), 30 mg/kg/day GED (GED), or 200 mg/kg/day CLA (CLA) for 5 days.

### DETAILED DESCRIPTION

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include to any animal, including mammals, preferably mice, rats, other rodents, rabbits, dogs, cats, swine, cattle, sheep, horses, or primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the invention is desirably a mammal in whom modulation of PPAR receptors is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system, animal or human that is being sought by the researcher, veterinarian, medical doctor or other clinician. The compounds of the invention are administered in therapeutically effective amounts to treat a disease. Alternatively, a therapeutically effective amount of a compound is the quantity required to achieve a desired therapeutic and/or prophylactic effect, such as an amount which results in the prevention of or a decrease in the symptoms associated with a disease associated with PPAR receptors.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts. Pharmaceutically acceptable salts of the disclosure include, for example, pharmaceutically acceptable salts of fatty acids, for example, pharmaceutically acceptable salts of conjugated linoleic acid.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein can exist in solvated as well as unsolvated forms with pharmaceutically acceptable solvents such as water, ethanol, and the like, and it is intended that the invention embrace both solvated and unsolvated forms. In one embodiment, the compound is amorphous. In one embodiment, the compound is a polymorph. In another embodiment, the compound is in a crystalline form.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the e.g., Examples herein by substituting an isotopically labeled reagent for a non-isotopically labeled reagent.

### Fatty Acids

The disclosure provides, at least in part, methods for treating, managing, preventing, and/or ameliorating lactose intolerance or a lactase deficiency by administering one or more isolated fatty acids to a patient, for example, a patient in need of treatment, prevention, management, and/or amelioration of lactose intolerance or a lactase deficiency. For example, in some embodiments, methods for treating, managing, preventing, and/or ameliorating lactose intolerance or a lactase deficiency include methods of administering a pharmaceutically acceptable composition, for example, a pharmaceutically acceptable formulation, that includes one or more isolated fatty acids, to a patient. Fatty acids and isolated fatty acids (used interchangeably herein) can refer to any fatty acid molecule or molecules that modulate a PPAR. Such fatty acids can, e.g., modulate PPAR activity, for example, by increasing PPAR activity such as by acting as a PPAR agonist or a PPARγ agonist. Without wishing to be bound by theory, a fatty acid can act as a PPAR modulator, for example, by binding to PPAR, for example, by acting as a PPAR ligand.

Fatty acids include, but are not limited to, saturated fatty acids, unsaturated fatty acids, short-chain fatty acids (e.g., fatty acids with aliphatic tails of fewer than six carbons), medium-chain fatty acids (e.g., fatty acids with aliphatic tails of 6-12 carbons), long-chain fatty acids (e.g., fatty acids with aliphatic tails of 13 to 21 carbons), linoleic acid, very long chain fatty acids (e.g., fatty acids with aliphatic tails longer than 22 carbons), omega-3 fatty acids, and essential fatty acids. Fatty acids also include isomers of fatty acids, for example, isomers of conjugated linoleic acid. Fatty acids also include isomers of fatty acids, for example, trans and cis isomers of fatty acids.

Unsaturated fatty acids include, for example, but are not limited to, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, stearidonic acid, γ-Linolenic acid, dihomo-γ-linolenic acid, docosatetraenoic acid, paullinic acid, gondoic acid, gadoleic acid, eicosenoic acid, nervonic acid, mead acid, crotonic acid, eicosadienoic acid, docosadienoic acid, pinolenic acid, elostearic acid, β-eleostearic acid, eicosatrienoic acid, eicosatetranoic acid, adrenic acid, bosseopentaenoic acid, ozubondo acid, sardine acid, herring acid, tetracosanolpentaenoic acid, and docosahexaenoic acid.

Saturated fatty acids include, for example, but are not limited to, propionic acid, butyric acid, valeric acid, caproic acid, caprylic acid, capric acid, lauric acid, myristic acid, palmitic acid, stearic acid, arachidic acid, behenic acid, lignoceric acid, cerotic acid, enanthic acid, pelargonic acid, undecylic acid, lauric acid, tridecylic acid, myristic acid, pentadecylic acid, margaric acid, nonadecylic acid, heneicosylic acid, tricosylic acid, pentacosylic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, and octatriacontanoic acid.

Fatty acids also include stereoisomers of fatty acids and racemic mixtures of fatty acid stereoisomers, for example, stereoisomers of linoleic acid, for example, 9(5)-hydroxy-10(E),12(Z)-octadecadienoic acid (9(S)-HODE) and 9(R)-hydroxy-10(E),12(Z)-octadecadienoic acid (9(R)-HODE), and racemic mixtures of linoleic acid stereoisomers, for example, 9-hydroxyoctadecadienoic acid (9-HODE). Other examples of stereoisomers of fatty acids include, but are not limited to 13-hydroxyoctadecadienoic acid (also known as 13-HODE, 13(S)-hydroxy-9Z,11E-octadecadienoic acid, or 13(S)-HODE) and 13(R)-hydroxy-9Z,11E-octadecadienoic acid (13(R)-HODE).

A fatty acid can be, e.g., a conjugated linoleic acid. Conjugated linoleic acid (CLA) refers to a group of positional and geometric isomers of linoleic acid that are characterized by the presence of conjugated dienes. A fatty acid can include any isomer of conjugated linoleic acid, including, e.g., the cis-9,trans-11 (c9,t11) isomer, trans-10,cis-12 (t10, c12) isomer, and trans-10,cis-11 (t10, c11) isomer. Exemplary conjugated linoleic acids are represented below the structure of linoleic acid:

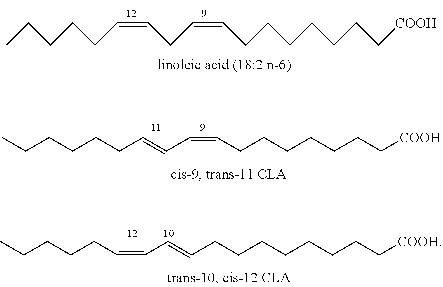 [SMILES: CCCCC/C=C\C/C=C\CCCCCCCC(=O)O ; CCCCCC/C=C/C=C\CCCCCCCC(=O)O ; CCCCC/C=C\C=C\CCCCCCCCC(=O)O]

The present disclosure also provides methods that include the use of pharmaceutical compositions comprising compounds as disclosed herein (e.g., an isolated CLA, as described above) formulated together with one or more pharmaceutically or cosmetically acceptable carriers. Exemplary compositions provided herein include compositions comprising essentially a CLA, as described above, and one or more pharmaceutically acceptable carriers. Formulations include those suitable for oral, rectal, topical, buccal, and parenteral (e.g., subcutaneous, intramuscular, intradermal, or intravenous) administration, or for topical use, e.g., as a cosmetic product. The most suitable form of administration in any given case will depend on the degree and severity of the condition being treated and on the nature of the particular compound being used.

In some embodiments, the disclosure is also directed to compositions for treating, preventing, monitoring, and/or ameliorating lactose intolerance and/or lactase deficiency that include one or more derivatives of fatty acids or products of fatty acid metabolism. In some embodiments, the disclosure is also directed to methods for treating, preventing, monitoring, and/or ameliorating lactose intolerance and/or lactase deficiency that include administering to a patient a composition that includes one or more derivatives of fatty acids or products of fatty acid metabolism. Derivatives of fatty acids and products of fatty acid metabolism include, for example, hormones such as prostaglandins (for example, 15-deoxy-delta-12,14-prostaglandin J2 (15d-PGJ2)), triglycerides, phospholipids, diacyl glycerols, second messengers (for example, inositol trisphosphate), and ketone bodies.

Derivatives of fatty acids include derivatives of linoleic acid, for example, DCP-LA (8-[2-(2-pentyl-cyclopropylmethyl)-cyclopropyl]-octanoic acid), FR236924, and oxidized derivatives of linoleic acid, including, but not limited to, 12,13-epoxy-9-keto-(10-trans)-octadecenoic acid (EKODE). Derivatives of fatty acids also include derivatives of arachidonic acid, including, but not limited to, 5-hydroxyicosatetraenoic acid (5-HETE), 12-hydroxyeicosatetraenoic acid (12-HETE), 15-hydroxyeicosatetraenoic acid (15-HETE), 16(R)-hydroxyeicosatetraenoic acid (16(R)-HETE), 16(S)-hydroxyeicosatetraenoic acid (16(S)-HETE), and 5(S),6(R)-Lipoxin A4, 5(S),6(R), and 15(R)-Lipoxin A4. Derivatives of fatty acids also include polyethylene glycol (PEG)ylated derivatives of fatty acids, for example pegylated derivatives of linoleic acid, for example, pegylated conjugated linoleic acid.

In some embodiments, the disclosure is directed to compositions for treating, preventing, monitoring, and/or ameliorating lactose intolerance and/or lactase deficiency that include one or more intermediate products of fatty acid metabolism, for example, intermediate products of linoleic acid metabolism. In some embodiments, the disclosure is also directed to methods for treating, preventing, monitoring, and/or ameliorating lactose intolerance and/or lactase deficiency that include administering to a patient a composition that includes one or more intermediate products of fatty acid metabolism, for example, intermediate products of linoleic acid metabolism. Intermediate products of linoleic metabolism include, for example, γ-linolenic acid, dihomo-γ-linolenic acid, arachidonic acid, and docosatetranoic acid.

In some embodiments, the disclosure is directed to compositions for treating, preventing, monitoring, and/or ameliorating lactose intolerance and/or lactase deficiency that include one or more fatty acid prodrugs, for example, a prodrug of conjugated linoleic acid. In some embodiments, the disclosure is also directed to methods for treating, preventing, monitoring, and/or ameliorating lactose intolerance and/or lactase deficiency that include administering to a patient a composition that includes one or more fatty acid prodrugs, for example, a prodrug of conjugated linoleic acid. As used herein, the term “prodrug” refers to a compound that is metabolized (e.g., metabolized after administration to a patient) into a pharmacologically active compound, for example, a pharmacologically active fatty acid. By way of example, prodrugs of conjugated linoleic acid include compounds that are metabolized to conjugated linoleic acid.

### Therapeutic Applications

The disclosure is directed at least in part to treating or ameliorating lactose intolerance or a lactase deficiency (or, e.g., controlling symptoms of lactose intolerance) by administering a fatty acid, e.g., a linoleic acid, e.g., a conjugated linoleic acid to a patient (e.g., a human patient) in need thereof. For example, methods of treating diarrhea, abdominal pain and/or bloating after lactose ingestion are provided, wherein a conjugated linoleic acid (or, e.g., a composition that includes a conjugated linoleic acid) is administered to a subject in need thereof, for example, by oral administration.

For example, in some embodiments, the disclosure is directed to methods of treating or ameliorating lactose intolerance or lactase deficiency in a patient by administering a fatty acid, e.g., a conjugated linoleic acid isomer, before, substantially simultaneously with, or after the patient ingests lactose, for example, a composition that includes lactose, for example, a food product that includes lactose.

Also provided herein are compositions for reducing lactose intolerance or lactase deficiency. For example, in some embodiments, a disclosed composition may form part of, or is used for making, a low lactose content milk or milk product, comprising a fatty acid, for example, a conjugated linoleic acid. Such compositions may be or may be part of, for example, a whey product, a milk product, or a cheese product.

Compounds of the invention may be administered to subjects (e.g., animals and/or humans) in need of such treatment or amelioration in dosages that will provide optimal pharmaceutical efficacy. It will be appreciated that the dose required for use in any particular application will vary from patient to patient, not only with respect to the particular compound or composition selected, but also with respect to the route of administration, the nature of the condition being treated, the age and condition of the patient, concurrent medication or special diets then being followed by the patient, and other factors which those skilled in the art will recognize, with the appropriate dosage ultimately being at the discretion of the attendant physician, caretaker, or patient. For treating clinical conditions and diseases noted above, compounds of this invention may be administered, for example, orally, topically, parenterally, by inhalation spray, or rectally in dosage unit formulations containing conventional, non-toxic, pharmaceutically acceptable carriers, adjuvants, and vehicles. The term parenteral as used herein includes subcutaneous injections, intravenous, intramuscular, intrasternal injection, or infusion techniques.

Generally, a therapeutically effective amount of active component will be in the range of from about 0.1 mg/kg to about 100 mg/kg, from about 0.1 mg/kg to about 1 mg/kg, from about 0.1 mg/kg to about 10 mg/kg, from about 1 mg/kg to about 100 mg/kg, from about 1 mg/kg to 10 mg/kg, from about 10 mg/kg to about 20 mg/kg, from about 20 mg/kg to about 30 mg/kg, from about 30 mg/kg to about 40 mg/kg, from about 40 mg/kg to about 50 mg/kg, from about 50 mg/kg to about 60 mg/kg, from about 60 mg/kg to about 70 mg/kg, from about 70 mg/kg to about 80 mg/kg, from about 80 mg/kg to about 90 mg/kg, or from about 90 mg/kg to about 100 mg/kg. The amount administered will depend on variables such as the type and extent of disease or indication to be treated, the overall health status of the particular patient, the relative biological efficacy of the compounds, formulations of compounds, the presence and types of excipients in the formulation, and the route of administration. The initial dosage administered may be increased beyond the upper level in order to rapidly achieve the desired blood-level or tissue level, or the initial dosage may be smaller than the optimum and the daily dosage may be progressively increased during the course of treatment depending on the particular situation. Human dosage can be optimized, e.g., in a conventional Phase I dose escalation study designed to run from 0.5 mg/kg to 20 mg/kg. Dosing frequency can vary, depending on factors such as route of administration, dosage amount, and the disease condition being treated. Exemplary dosing frequencies are once per day, once per week and once every two weeks.

Formulations or compositions of the disclosure can comprise a disclosed compound and typically can also include a pharmaceutically acceptable carrier or excipient.

Compositions of the disclosure may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular, or subcutaneous), drop infusion preparations, enemas, or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In some embodiments of the formulations provided herein, wetting agents, emulsifiers, and lubricants, such as sodium lauryl sulfate and magnesium stearate, as well as coloring agents, release agents, coating agents, sweetening, flavoring, perfuming agents, preservatives, and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose may vary depending upon the subject being treated, and the particular mode of administration.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, film-coated tablets, sugar-coated tablets, powders, granules and the like), compositions of the disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration may include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Throughout the description, where compositions are described as having, including, or comprising specific components, it is contemplated that compositions also consist essentially of, or consist of, the recited components. Similarly, where processes are described as having, including, or comprising specific process steps, the processes also consist essentially of, or consist of, the recited processing steps.

Except where indicated otherwise, the order of steps or order for performing certain actions are immaterial so long as the invention remains operable. Moreover, unless otherwise noted, two or more steps or actions may be conducted simultaneously.

The compounds disclosed herein can be prepared in a number of ways well known to one skilled in the art of organic synthesis.

### Pharmaceutical Compositions and Routes of Administration

The present disclosure also provides methods for treating, preventing, or ameliorating lactose intolerance or lactase deficiency by administering a pharmaceutical composition comprising one or more isolated fatty acids, e.g., a conjugated linoleic acid (CLA), for example, a trans-10, cis-12 conjugated linoleic acid isomer, a cis-9, trans-11 conjugated linoleic acid isomer, or a mixture thereof. In another aspect, the disclosure provides pharmaceutical compositions for use in treating lactose intolerance or lactase deficiency. Pharmaceutical compositions may be comprised of a disclosed isolated fatty acid, for example, a CLA, and a pharmaceutically acceptable carrier. In embodiments, a pharmaceutical composition may be a mixture containing a specified amount of a therapeutic compound, e.g., a therapeutically effective amount, of a therapeutic compound, for example, a therapeutically effective amount of a fatty acid (e.g., a CLA), in a pharmaceutically acceptable carrier for administering to a patient, e.g., a human, in order to treat, manage, ameliorate, and/or prevent lactose intolerance or lactase deficiency. In some embodiments, provided herein are pharmaceutical compositions comprising a disclosed isolated fatty acid and a pharmaceutically acceptable carrier. In some embodiments, the disclosure is directed to use of a isolated fatty acid in the manufacture of a medicament for treating, managing, ameliorating, and/or preventing lactose intolerance or a lactase deficiency. “Medicament,” as used herein, has essentially the same meaning as the term “pharmaceutical composition.”

Pharmaceutically acceptable carriers may include buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating or a lipophilic coating suitable for regulating the site of absorption of the encapsulated substances within the digestive system or gut. For example, an enteric coating can include an ethylacrylate-methacrylic acid copolymer, an amino alkyl methacrylate copolymer, a methacrylic acid copolymer, a methacrylic ester copolymer, an ammonioalkyl methacrylate copolymer, a polymethacrylate, a poly(methacrylic acid-co-methyl methacrylate), hydroxypropyl-methylcellulose phthalate.

In some embodiments, formulations provided herein include enteric coatings, for example, lipophilic coatings, that allow delivery of a therapeutic, for example, an isolated fatty acid, to one or more specific regions of the gastrointestinal tract. For example, formulations may include enteric coatings and reagents that allow delivery of therapeutic to the stomach, the duodenum, the jejunum, the small intestine, the large intestine, the transverse, ascending, or descending colon, the ileum, the cecum, and/or the rectum. Formulations may include enteric coatings and reagents that allow release of therapeutic from a formulation for oral administration in the form of, for example, a tablet, a lozenge, or a capsule, at an approximate pH value or within a pH value range. For example, formulations provided herein may include enteric coatings and reagents that release therapeutic, for example, an isolated fatty acid, from a formulation for oral administration at a pH value of about 3, about 4, about 4.5, about 5, about 5.5, about 6, about 6.5, about 7, about 7.5, or about 8. For example, formulations provided herein may include enteric coatings and reagents that release therapeutic from a formulation for oral administration at a pH value of greater than about 3, greater than about 4, greater than about 4.5, greater than about 5, greater than about 5.5, greater than about 6, greater than about 6.5, greater than about 7, greater than about 7.5, or greater than about 8. In some embodiments, formulations of the disclosure release therapeutic from a formulation for oral administration in a pH value range of about pH 3 to about pH, about pH 4 to about pH 5, about pH 5 to about pH 6, about pH 6 to about pH 7, about pH 7 to about pH 8, about pH 8 to about pH 9, about pH 4.5 to about pH 7.5, about pH 4 to about pH 7, about pH 5 to about pH 7, about pH 5.5 to about pH 6.5, or about pH 4.5 to about pH 5.5.

In some embodiments, a disclosed fatty acid and any pharmaceutical composition thereof may be administered by one or several routes, including topically, parenterally, orally, pulmonarily, intratracheally, intranasally, transdermally, or intraduodenally. Parenteral administration includes subcutaneous injections, intrapancreatic administration, intravenous, intramuscular, intraperitoneal, intrasternal injection or infusion techniques. For example, a fatty acid may be administered subcutaneously to a subject. In another example, a fatty acid may be administered orally to a subject. In another example, a fatty acid may be administered directly to the gastrointestinal system, or specific regions of the gastrointestinal system (e.g., the ileum, colon, or rectum) via parenteral administration.

Pharmaceutical compositions containing a fatty acid, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

### Parenteral Administration

The pharmaceutical compositions of the disclosure can be formulated for parenteral administration, e.g., formulated for injection via the intravenous, intramuscular, subcutaneous, intralesional, or intraperitoneal routes. The preparation of an aqueous composition, such as an aqueous pharmaceutical composition containing a fatty acid, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used (beyond their use as therapeutic agents) in the preparation of injectables. Sterile injectable preparations may also be sterile injectable solutions, suspensions, or emulsions in a nontoxic parenterally acceptable diluent or solvent, for example, as solutions in 1,3-butanediol. Among the acceptable vehicles and solvents that may be employed are water, Ringer's solution, U.S.P., and isotonic sodium chloride solution. In a particular embodiment, a fatty acid may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a fatty acid in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of fatty acid to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium 10 carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose , petrolatum, polyethylene glycol, polyvinyl alcohol, polyvinylpyrrolidone, carboxymethylcellulose and the like.

### Oral Administration

In some embodiments, provided herein are compositions suitable for oral delivery of a fatty acid, e.g., tablets that include an enteric coating, e.g., a gastro-resistant coating, such that the compositions may deliver fatty acid to, e.g., the gastrointestinal tract of a patient. For example, such administration may result in a topical effect, substantially topically applying the fatty acid directly to an affected portion of the gastrointestinal tract of a patient. Such administration, may, in some embodiments, substantially avoid unwanted systemic absorption of a fatty acid.

For example, a tablet for oral administration is provided that comprises granules (e.g., is at least partially formed from granules) that include a fatty acid, e.g., an isolated naturally occurring fatty acid, e.g., a trans-10, cis-12 conjugated linoleic acid isomer, a cis-9, trans-11 conjugated linoleic acid isomer, or a mixture of one or more conjugated linoleic acids, and one or more pharmaceutically acceptable excipients. Such a tablet may be coated with an enteric coating. Tablets provided herein may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, provided pharmaceutical formulations include an intra-granular phase that includes a fatty acid, e.g., an isolated naturally occurring fatty acid, e.g., a trans-10, cis-12 conjugated linoleic acid isomer, a cis-9, trans-11 conjugated linoleic acid isomer, or a mixture of one or more conjugated linoleic acids, and a pharmaceutically acceptable salt, e.g., a disclosed fatty acid, e.g., an isolated naturally occurring fatty acid, e.g., a trans-10, cis-12 conjugated linoleic acid isomer, a cis-9, trans-11 conjugated linoleic acid isomer, or a mixture of one or more conjugated linoleic acids, and a pharmaceutically acceptable filler. For example, a disclosed fatty acid and a filler may be blended together, optionally, with other excipients, and formed into granules. In some embodiments, the intragranular phase may be formed using wet granulation, e.g. a liquid (e.g., water) is added to the blended fatty acid compound and filler, and then the combination is dried, milled and/or sieved to produce granules. One of skill in the art would understand that other processes may be used to achieve an intragranular phase.

In some embodiments, provided formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or a extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Formulations of the disclosure, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a provided formulation includes an intra-granular phase comprising a fatty acid and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a provided formulation may include a lubricant, e.g., an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, a pharmaceutical formulation comprises an enteric coating, for example, a lipophilic coating. Generally, enteric coatings create a barrier for the oral medication that controls the location at which the drug is absorbed along the digestive tract. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a tablet by weight. For example, enteric coatings may include an ethylacrylate-methacrylic acid copolymer.

For example, in some embodiments provided herein, a tablet is provided that comprises or consists essentially of about 0.5% to about 70%, e.g. about 0.5% to about 10%, or about 1% to about 20%, by weight of a fatty acid or a pharmaceutically acceptable salt thereof. Such a tablet can include, for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a fatty acid, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In another embodiment, a pharmaceutical tablet formulation for oral administration of a fatty acid comprises an intra-granular phase, wherein the intra-granular phase includes a fatty acid or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may include about 0.5% to 10% by weight of a disclosed fatty acid, e.g., a CLA or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a fatty acid, e.g., a CLA, or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO2010/054826, which is hereby incorporated by reference in its entirety).

The rate at which point the coating dissolves and the active ingredient is released is its dissolution rate. In an embodiment, a tablet may have a dissolution profile, e.g. when tested in a USP/EP Type 2 apparatus (paddle) at 100 rpm and 37° C. in a phosphate buffer with a pH of 7.2, of about 50% to about 100% of the fatty acid releasing after about 120 minutes to about 240 minutes, for example after 180 minutes. In another embodiment, a tablet may have a dissolution profile, e.g. when tested in a USP/EP Type 2 apparatus (paddle) at 100 rpm and 37° C. in diluted HC1 with a pH of 1.0, where substantially none of the fatty acid is released after 120 minutes. A tablet provided herein, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the fatty acid releasing after 30 minutes.

Formulations, e.g., tablets, in some embodiments, when orally administered to the patient may result in minimal plasma concentration of the fatty acid in the patient. In another embodiment, disclosed formulations, when orally administered to a patient, topically deliver to the colon or rectum of a patient, e.g., to an affected or diseased site of a patient.

In some embodiments, methods provided herein may further include administering at least one other agent that is directed to treatment of diseases and disorders disclosed herein. In one embodiment, contemplated other agents may be co-administered (e.g., sequentially or simultaneously).

Agents contemplated include immunosuppressive agents including glucocorticoids, cytostatics, antibodies, agents acting on immunophilins, interferons, opioids, TNF binding proteins, mycophenolate, and small biological agents. For example, contemplated immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, certolizumab pegol, natalizumab, and etanercept. Other contemplated agents include antibiotics, anti-diarrheals, laxatives, pain relievers, other fatty acids, iron supplements, and calcium or vitamin D or B-12 supplements.

### Dosage and Frequency of Administration

Exemplary formulations include dosage forms that include or consist essentially of about 35 mg to about 500 mg of a fatty acid. For example, formulations that include about 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, or 250 mg of a fatty acid are provided herein. In one embodiment, a formulation may include about 40 mg, 80 mg, or 160 mg of a fatty acid. In some embodiments, a formulation may include at least 100 μg of a fatty acid. For example, formulations may include about 0.1 mg, 0.2 mg, 0.3 mg, 0.4 mg, 0.5 mg, 1 mg, 5 mg, 10 mg, 15 mg, 20 mg, or 25 mg of a fatty acid. The amount administered will depend on variables such as the type and extent of disease or indication to be treated, the overall health and size of the patient, the in vivo potency of the fatty acid, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 40 mg to 160 mg. Dosing frequency can vary, depending on factors such as route of administration, dosage amount and the disease being treated. Dosing frequencies can include once per day, twice per day, 3 times per day, 4 times per day, 5 times per day, 6 times per day, 7 times per day, 8 times per day, 9 times per day, 10 times per day, more than 10 times per day, once per week, once every two weeks, once per month, and as needed. In some embodiments, dosing is once per day for 7 days.

### EXAMPLES

The embodiments described herein are further illustrated by the following examples. The examples are provided for illustrative purposes only, and are not to be construed as limiting the scope or content of the embodiments in any way.

### Example 1 Materials and Methods

### Cell Culture and Treatment

Caco-2 (colonic adenocarcinoma) cells were grown in Dulbecco's Modified Eagle's Medium (DMEM, Invitrogen, Life Technologies, Cergy-Pontoise, France) supplemented with 20% foetal calf serum (FCS, Dutscher, Brumath, France), 1% penicillin-streptomycin (5 ml/l) (Invitrogen, Life technologies) and 1% non-essential amino acids (5 ml/l) (Invitrogen, Life technologies). All cell lines were cultured as confluent monolayers at 37° C. in a controlled, 5% CO₂ atmosphere.

For cell stimulations, 1×10⁶ cells per well were seeded in 6-well plates. Cells were serum deprived for 16 hours prior to stimulation in order to synchronize the cells. Cells were treated with GED (Nogra Pharma Ltd, Ireland), pioglitazone (1 μM, Sigma-Aldrich) or CLA (various concentrations, Sigma-Aldrich). When necessary, the DMSO vehicle (Sigma-Aldrich) was used as control. After 24 hours of stimulation, cells were washed three times with sterile PBS before RNA extraction. Cell stimulations were performed in 4 replicates for microarray analysis and in 3, 4 or 6 replicates for other stimulations.

### RNA Extraction

Cells were lysed with lysis buffer (RA1, Macherey-Nagel) containing 1% β-mercaptoethanol. Total RNA was extracted with a Nucleospin RNA kit (Macherey-Nagel, Hoerdt, France). After RNAse inactivation, total RNA was cleaned of genomic DNA traces by DNAse treatment and eluted in RNAse-free, DEPC-water. The purity of the RNA was evaluated by UV spectroscopy on a Nanodrop system (Nyxor Biotech, Paris, France) from 220 to 350 nm. Before microarray experiments, RNAs were also profiled on an Agilent 2100 bioanalyzer. One μg of total RNA with a minimum concentration of 50 ng/μl was used in the microarray and qRT-PCR analysis.

### Microarrays

Dual-colour gene expression microarrays were used to compare the cRNA from the samples. 44,000 genes were screened. The RNAs from the samples were first reverse-transcribed into cDNA (Affinity-Script RT, Agilent), which were then used as the substrate for the synthesis and amplification of cRNA by T7 RNA polymerase in the presence of cyanine 3-CTP for the CTL sample (green fluorescence) and cyanine 5-CTP for the PPAR-γ y modulator sample (red fluorescence). The two-labelled cRNAs were mixed, hybridized on the same array (G4851A Agilent 8×44K) and then scanned (with an Agilent G2505B scanner). Fluorescence was visualized after laser excitation and the relative intensities of the two fluorophores were expressed as a ratio, in order to yield the over- or under-expression status of each gene (using GeneSpring software (Agilent)). This analysis was performed for each PPAR-γ modulator.

### Quantitative PCR

Expression of genes of interest was quantified by quantitative PCR (qPCR) of corresponding reverse transcribed mRNA. One μg of total RNA was reverse-transcribed into cDNA using the High Capacity cDNA Archive kit (Applied Biosystems). Amplification was performed using an ABI PRISM 7000 sequence detection system (Applied Biosystem) using Power SYBR® Green PCR master Mix (Applied Biosystem). Primer pairs for each human transcript were chosen with qPrimer depot software (at primerdepot.nci.nih.gov.). Quantification of qPCR signals was performed using ΔCt relative quantification method using GAPDH as a reference gene for human and rat samples and β-Actin for mouse samples. Values were represented in terms of relative quantity of mRNA level variation or fold increase compared to control conditions.

### Immunoprecipitation

LCT protein expression level was determined by immunoprecipitation followed by Western Blotting analysis. Briefly, total proteins were extracted from Caco-2 cells using a RIPA Buffer containing 25 mM Tris-HCl pH 7.6, 150 mM NaCl, 1% NP-40, 1% Sodium deoxycholate, 0.1% Sodium Dodecyl sulphate supplemented with classical protease-inhibitor cocktail. 250 μg of total protein were immunoprecipitated with 2 μg of a specific antibody against Lactase (Santa Cruz) overnight at 4° C. The immunoprecipitated proteins were coated with protein A/G Agarose beads (Santa Cruz) and mixed gently for 4 hours at 4° C. Beads were washed three times in RIPA buffer and then electrophoresed through a 12.5% SDS-PAGE and transferred onto polyvinylidene fluoride membranes (PVDF; Amersham Biosciences). Membranes were immunoblotted with a specific monoclonal antibody against LCT (Cell Signaling; 1:1000 overnight at 4° C.) or β-Actin (Sigma Aldrich; 1:20000 for 2 hours at room temperature) for the “10% input” loading control. Membranes were then incubated with secondary horseradish peroxidase-conjugated antibodies (anti-rabbit (Jackson ImmunoResearch) and anti-mouse (Sigma), 1:10000 for 1 hour at room temperature) and finally revealed with chemiluminescent substrate according to the manufacturer's protocol (ECL; Millipore Corporation). Membranes were exposed to autoradiography films (Hyperfilm; Amersham Biosciences).

### Lactase Activity

Lactase activity was evaluated by using a glucose oxidase method (Glucose Assay Kit, Sigma). This lactase assay is based on the measurement of the amount of glucose produced following the action of lactase by incubating samples with a lactose buffer solution (0.056 mol/l lactose in a 0.1 mol/l Na-maleate buffer). For Caco-2 cells, lactase activity was determined directly from the cell monolayer. After extensive washing, the cell monolayer was incubated with lactose buffer for one hour at 37° C. The supernatant was recovered, 50 μl were diluted with 100 μl of glucose oxidase reagent and incubated at 37° C. for 1 hour. The reaction was stopped with 100 μl of H₂SO₄ and read by spectrophotometry at 450 nm. When lactase activity was determined from intestinal sample, tissue samples were first dounce-homogenized in 0.9% NaCl on crushed ice. These homogenates were then diluted in 0.9% NaCl ( 1/500) and 50 μl of dilution were incubated with lactose buffer and used to determine lactase activity. For each experiment, the background attributed to the remaining glucose in the samples was measured by incubating cells or cell extracts in lactose-free buffer.

### Glucose Uptake Assay

Glucose uptake was evaluated by using the glucose uptake colorimetric assay kit (Sigma-Aldrich) according to the manufacturer's instructions. Briefly, Caco-2 cells were seeded into a 96-well plate at a density of 30,000 cells per well. Cells were serum deprived for 16 hours prior to stimulation in order to synchronize the cells. Cells were treated with GED (1 mM) or pioglitazone (1 μM) for 24 h. Cells were then washed 3 times with PBS and were glucose-starved by incubating with 100 μl of KRPH buffer (Krebs-Ringer-Phosphate-HEPES (KRPH) Buffer—20 mM HEPES, 5 mM KH2PO4, 1 mM MgSO4, 1 mM CaCl2, 136 mM NaCl, and 4.7 mM KCl, pH 7.4) containing 2% BSA for 40 minutes. 10 μl of 2-deoxyglucose (2-DG; 10 mM) was then added and incubation was continued for 20 minutes. 2-DG is taken up by the cells and phosphorylated by hexokinase to 2-DG6P, which cannot be further metabolized and accumulates in cells. Following incubation, cells were washed 3 times with PBS and lysed with 80 μl of the extraction buffer provided. The amount of 2-DG6P (which is directly proportional to glucose uptake by the cells) was determined by a colorimetric detection assay according to the manufacturer's protocol.

### Generation of PPAR-γ Knock-down Cells

PPARγ knockdown IECs were obtained using the pSUPER.retro system (OligoEngine). Forward and reverse target sequences corresponding to nucleotides 105-123 of the human PPARγ mRNA (5′-GCCCTTCACTACTGTTGAC-3′ (SEQ ID NO: 1)) were cloned into the BgllI/XhoI restriction sites of the pSUPERretro vector (pRS) giving the ShPPAR construct. A negative control pRS plasmid containing the sequence 5′-ACGCTGAGTACTTCGAAAT-3′ (SEQ ID NO: 2) targeted against the luciferase gene was also generated (ShLuc construct). Both constructions were transfected in Caco-2 cells using Nucleofector technology from Amaxa Biosystems, according to the manufacturer's protocol. Stably transfected clones were selected 24 h post-transfection with complete culture medium supplemented with puromycin (5 μg/ml). The silencing of PPARγ expression was checked by quantitative RT-PCR and western-blot analysis. Once established, ShPPAR and ShLuc cell lines were maintained in complete medium supplemented with 2.5 μg/ml puromycin.

### Reporter Gene Assay

A 321 bp genomic fragment (corresponding to the first 321 bp upstream to the transcription start site of the human lactase gene) was cloned in the pGL4-Luc reporter vector using XhoI and Hind III restriction sites introduced in “Hs-Prom-0.3 Kb sens” and “Hs-Prom-0.3 Kb anti-sens” oligonucleotides respectively. This construct and the empty vector control were transiently transfected in Caco-2 cells using Nucleofector™ Technology. Six hours post-transfection, cells were treated with PPARγ modulator for 12 hours. Luciferase activity was measured using the luciferase assay kit (Promega) in a Wallac Victor2 ™ 1420 multilabel counter (Perkin Elmer).

### Chromatin Immunoprecipitation Experiments

The physical binding of PPARγ onto the LCT gene promoter was studied by Chromatin immunoprecipitation (ChIP) experiments in Caco-2 cells (5×10⁶ cells) stimulated for 24 hours with 1 mM GED in 100 mm cell culture petri dishes. Caco-2 cells were synchronized by the addition of serum-free medium for 16 hours and then stimulated for 24 hours using the protocol described previously. Cells were then rinsed with PBS and the protein-DNA complexes were fixed by adding 1% PFA for 30 minutes at room temperature. This binding was stopped by the addition of glycine (0.125M). Cells were collected by scraping in the presence of cold PBS and protease inhibitors (Sigma). The cell pellet obtained by centrifugation was taken up in 300 μl SDS buffer (1% SDS, 10 mM EDTA, 50 mM Tris-HCl pH 8, protease inhibitors) and sonicated (Diagenode, BioruptorUCD200TM-EX) for 30 seconds, followed by 30 seconds resting time. For each immunoprecipitation, 125 μL of crosslinked sonicated sample was diluted with 225 μl of IP buffer (1% triton X-100, 150 mM NaCl, 2 mM EDTA, 20 mM Tris-HCl pH 8.1 and protease inhibitors) and pre-cleared for four hours by adding 40 μL of protein A/G beads (50% slurry protein A/G Sepharose, Clinisciences) and 5 μg of salmon sperm DNA (Invitrogen). Complexes were immunoprecipitated with specific anti-PPARγ antibodies (C26H12 rabbit monoclonal antibody, CellSignaling) by incubation overnight at 4° C. under rotation. Immune complexes were recovered by adding 40 μL of protein A/G Sepharose (50%) plus 2 μg salmon sperm DNA and incubated for four hours at 4° C. The beads were washed twice in wash buffer 1 (0.1% SDS, 1% Triton X-100, 150 mM NaCl, 0.1% Deoxycholate, 1 mM EGTA, 2 mM EDTA, 20 mM Tris HCl pH 8.0), twice in wash buffer 2 (0.1% SDS, 1% Triton X-100, 500 mM NaCl, 0.1% Deoxycholate, 1 mM EGTA, 2 mM EDTA, 20 mM Tris HCl pH 8.0), once in wash buffer 3 (0.25 mM LiCl, 0.5% Deoxycholate, 0.5% NP40, 0.5 mM EGTA, 1 mM EDTA, 10 mM Tris HCl pH 8.0) and 3 times in wash buffer 4 (1 mM EDTA, 10 mM Tris HCl pH 8.0). The co-immunoprecipitated DNA was then extracted with 150 μl of extraction buffer (0.1M NaHCO3, 1% SDS). Cross-linking was reversed overnight at 65° C. DNA was then purified using the PCR Clean-up kit (Macherey-Nagel) and analyzed by PCR.

### Animal Experimentation

Animal experiments were performed in the accredited Pasteur Institute animal care facility (Institut Pasteur de Lille, France; n° B59-35009) according to governmental guidelines (n° 2010/63/UE; Décret 2013-118) and animal ethics committee approval. Specific pathogen-free male C57BL/6 mice and Sprague-Dawley rats were obtained from Janvier Labs (France). Mice and rats were housed 5 animals/cage and 3 animals/cage, respectively, in a specific pathogen-free facility, in an air-conditioned room with controlled temperature (22±1° C.), humidity (65-70%), and 12 h light/12 h dark cycles. Animals were fed with standard laboratory chow (except when indicated) and were provided with autoclaved tap water ad libitum. Animals were acclimatized for at least 1 week before entering the study.

In order to assess the effect of GED on lactase expression and activity, weaned C57BL/6 mice (8 weeks old) and weaned Sprague-Dawley rats (older than two months) were randomized to 2 groups receiving daily intragastric gavage of 30 mg/kg of GED or vehicle (0.5% CMC, 1% Tween 80). After 7 days of treatment, animals were euthanized and the gastrointestinal tract was removed via a midline laparotomy. Approximately 0.5 cm of proximal intestine tissue specimens were snap frozen for further extractions. LCT mRNA expression and LCT activity were assessed as described above.

In addition, the effect of GED on the symptoms associated with lactose intolerance was evaluated in weaned rats fed with a lactose-enriched diet provided by Ssniff Spezialdiäten GmbH (Soest, Germany). Animals were monitored daily, weighed, and stool consistencies were evaluated.

Proximal intestine samples from knockout mice harbouring a specific PPAR-γ deletion in IEC (PPAR-γΔ^{IEC}) were provided by Prof. Daniel Metzger (Institute of Genetics and Molecular and Cellular Biology IGBMC (Inserm/CNRS/University of Strasbourg)).

### SCFA Quantification

SCFA were extracted and measured as described in Momose, Y., et al. “Studies on antidiabetic agents X. Synthesis and biological activities of pioglitazone and related compounds.” Chem Pharm Bull (Tokyo) 39, 1440-1445 (1991), which is incorporated herein by reference.

### Genotyping

LCT genotyping of C/T13910 and G/A22018 polymorphisms for Caco-2 cells were determined as described in Mastrofrancesco, A., et al. “Preclinical studies of a specific PPARgamma modulator in the control of skin inflammation.” J Invest Dermatol 134, 1001-1011 (2014), which is incorporated herein by reference.

### Statistical Analysis

All graphs were plotted and analysed with GraphPad Prism 5 Software (GraphPad Software, San Diego, Calif.) and StatXact v.7.0 (Cytel Studio) software using a nonparametric Mann-Whitney test. P values of less than 0.05 (P<0.05) were considered statistically significant.

### Example 2: PPARγ Modulators Induce Lactase Activity

In order to determine what gene expression changes were induced by PPARγ activation, gene expression data was collected from unstimulated Caco-2 cells and from Caco-2 cells following PPARγ stimulation. Gene expression profile changes in Caco-2 cells following exposure to PPARγ modulators were evaluated by microarray analysis. Two different PPARγ activators were used in the study: pioglitazone (Pio) and GED-0507-34-Levo (GED). Pioglitazone (Pio) is a well-known member of the TZD drug class. GED is a member of the aminophenyl-alpha-methoxypropionic acid family of compounds. GED is described in U.S. patent application Ser. No. 14/394,916, which is hereby incorporated by reference in its entirety. Among the 44,000 genes analyzed, it was observed that the LCT gene was the leading gene upregulated following stimulation with 1 mM GED, 30 mM GED, and 1 μM Pio (Table 1). LCT gene expression was significantly increased in response to 1 mM GED (5.28-fold±0.55; P<0.05), 30 mM GED (8.28-fold±1.7; P<0.05), and by Pio (17.93-fold±5.1; P<0.05) compared to unstimulated cells.

TABLE 1
Analysis of LCT mRNA expression in transcriptome microarray
data from IECs treated with different PPARγ modulators

| Stimulation | Fold Lactase RNA | Rank |
|---|---|---|
| GED 1 mM | 5.29 | 1/46 |
| GED 30 mM | 8.28 | 1/355 |
| Pioglitazone 1 μM | 17.9 | 1/121 |
| 5ASA 30 mM | 8.7 | 16/1574 |

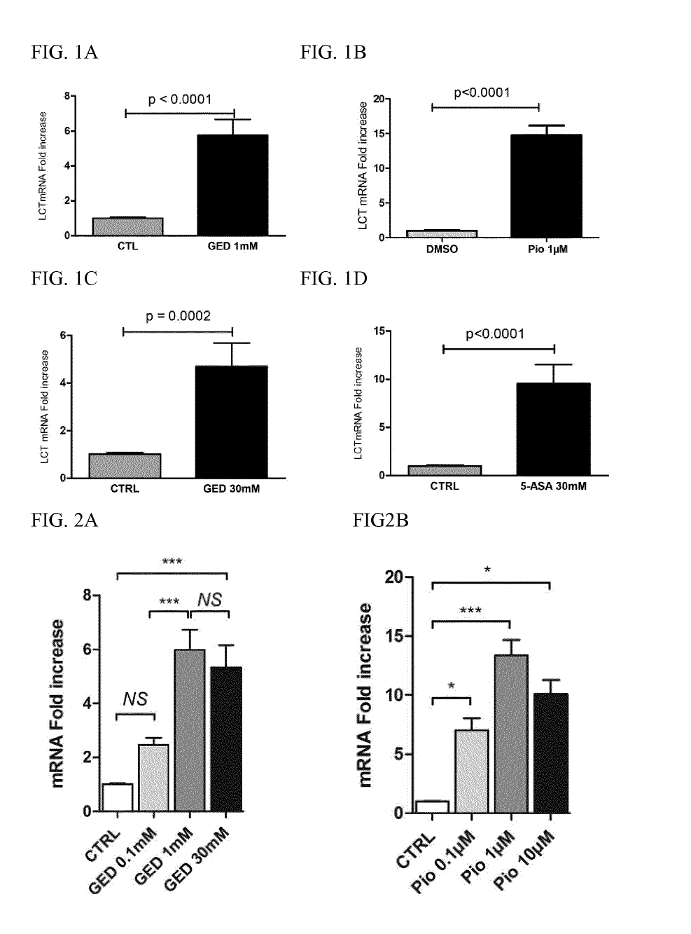

The ability of GED and pioglitazone to stimulate LCT gene expression was confirmed by evaluating LCT gene expression in Caco-2 cells using quantitative RT-PCR (qRT-PCR) following exposure to GED at 1 mM and 30 mM, Pio at 1 μm, and 5-aminosalicylic acid (5-ASA). Significant increases in LCT mRNA expression relative to control levels were observed following exposure to 1 mM GED (5.76±0.89-fold change; p<0.0001; FIG. 1A), 1 μM Pio (14.77±1.37-fold change; p<0.0001; FIG. 1B), 30 mM GED (p=0.0002; FIG. 1C), and 30 mM 5-ASA (p<0.0001; FIG. 1D).

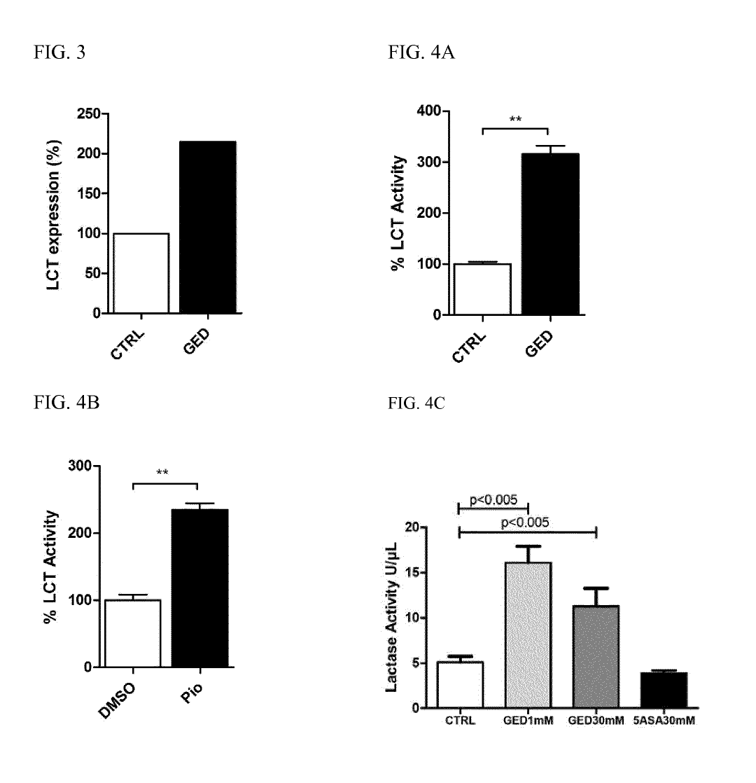

Changes in LCT mRNA expression levels in Caco-2 cells in response to increasing levels of the PPARγ activator GED were also analyzed. Dose-response analyses demonstrated that LCT mRNA gene expression was increased relative to unstimulated cells (CTRL) following exposure to GED at 0.1 mM, 1 mM, and 30 mM (FIG. 2A) or exposure to Pio at 0.1 μM, 1 μM, or 10 μM (FIG. 2B). The largest mean increases in LCT gene expression were observed following exposure to 1 mM GED and 1 μM Pio (FIG. 2A and 2B). Immunoprecipitation and immunostaining assays also demonstrated that PPARγ activators induced an increase in LCT protein expression in Caco-2 cells (FIG. 3 and data not shown). These results demonstrate that stimulation of PPARγ by agonist compounds resulted in a significant and robust increase in LCT mRNA and protein expression levels.

To determine whether PPARγ stimulation results in increased LCT activity, LCT activity in Caco-2 cells was measured following stimulation with PPARγ modulators. LCT activity was measured as the rate of glucose production in Caco-2 culture supernatant following incubation of Caco-2 cells with lactose. Stimulation of Caco-2 cells by 1 mM GED (FIG. 4A) or 1 μM Pio (FIG. 4B) significantly increased LCT activity compared to untreated cells (CTRL or DMSO, FIGS. 4A and 4B, respectively) by more than 3-fold and 2-fold, respectively (FIGS. 4A and 4B). LCT activity was also evaluated following stimulation of Caco-2 cells with 1 mM GED, 30 mM GED, or 30 mM 5-ASA. A significant increase in LCT activity was observed relative to control cells (CTRL) following stimulation with 1 mM or 30 mM GED, but not with 30 mM 5-ASA (FIG. 4C; CTRL v. 1 mM GED, p<0.005; CTRL v. 30 mM GED, p<0.005). These results demonstrate that stimulation of an intestinal epithelial cell line with multiple PPARγ agonists resulted in significant increases in LCT activity.

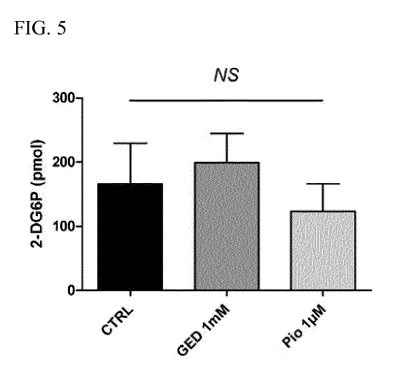

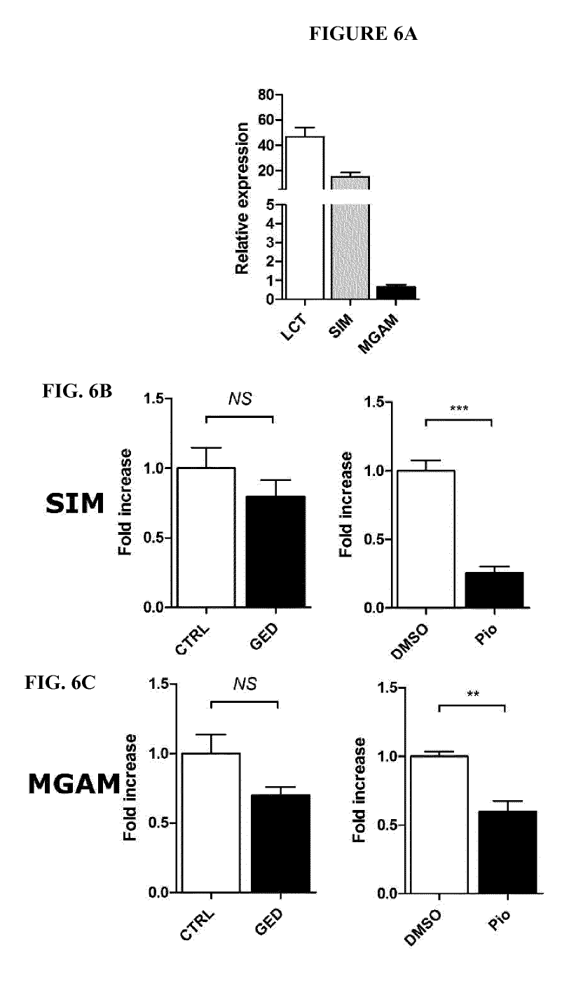

Stimulation of Caco-2 cells with 1 mM GED or 1 μM Pio did not significantly alter glucose uptake of Caco-2 cells, despite the observed increase in LCT expression and activity (FIG. 5). Expression levels of the disaccharidases sucrase-isomaltase (SIM) and maltase-glucoamylase (MGAM) were found to be lower than those of LCT in Caco-2 cells. Additionally, while PPARγ stimulation increased LCT gene expression, no significant increase in expression of SIM or MGAM was observed following PPARγ stimulation (FIGS. 6B and 6C). In fact, pioglitazone stimulation induced a significant decrease in both SIM and MGAM expression (FIGS. 6B and 6C). These experiments demonstrate that the observed PPARγ agonist induced increase in glucose production did not occur as a result of increased expression of disaccharidases other than LCT or as a result of increased glucose uptake by Caco-2 cells. Rather, these results strongly suggest that any increase in glucose production resulted from increased LCT gene expression following PPARγ agonist stimulation.

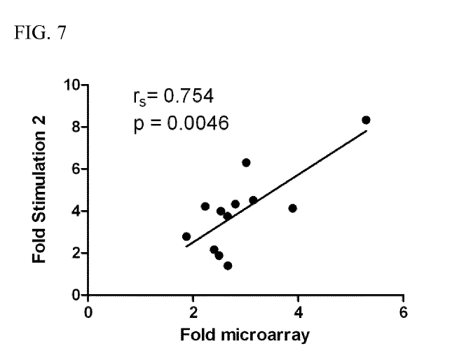

To evaluate the reproducibility of microarray data obtained from Caco-2 cells stimulated with PPARγ agonists, a correlative analysis of data obtained from quantitative PCR (qPCR) and microarray studies was performed (FIG. 7). This analysis confirmed that a significant correlation exists between the microarray (Fold microarray) and qPCR (Fold Stimulation 2) data (r=0.754, p=0.0046).

Altogether, these data demonstrate that PPARγ modulators were able to induce LCT mRNA expression and LCT activity in Caco-2 cells.

### Example 3: Analysis of the Lactase Gene Promoter

Among the single nucleotide polymorphisms characterized in the human LCT gene, two polymorphisms, C/T13910 and G/A22018, are linked to hypolactasia. The homozygous CC13910 and GG22018 genotypes are associated with the lactase non-persistent phenotype. Interestingly, the genotype of Caco-2 cells is CC13910 and GG22018, suggesting that PPARγ modulators may be able to control LCT gene expression in lactase non-persistent individuals.

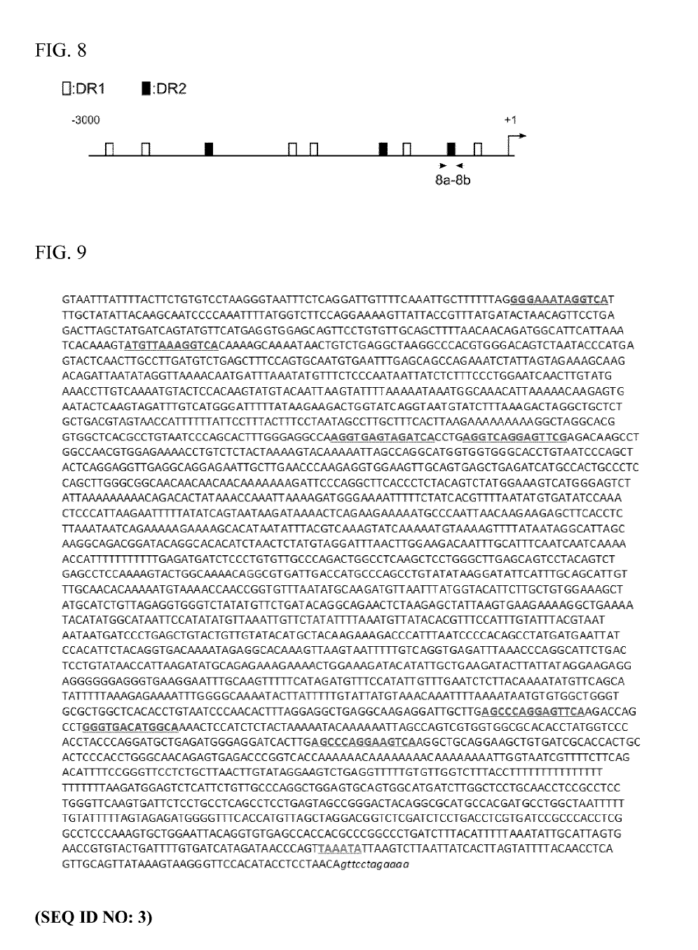

To investigate this possibility, the LCT gene promoter was analyzed for the presence of PPAR response element (PPRE) sequences. PPARγ is able to bind DNA as a heterodimer with another nuclear receptor RXR. The heterodimer PPARγ-RXR recognizes short dimeric palindromic sequences (consensus sequence AGGTCA or TGACCT) spaced by one nucleotide (known as direct repeat 1 (DR1)) or two nucleotides (known as direct repeat 2 (DR2)), which define the PPRE. In silico analysis of the 3,000 base pairs upstream from the transcription start site of the human LCT gene was performed and revealed the presence of several potential DR1 and DR2 PPRE sequences, that could allow PPARγ to regulate LCT gene expression (FIG. 8 and FIG. 9).

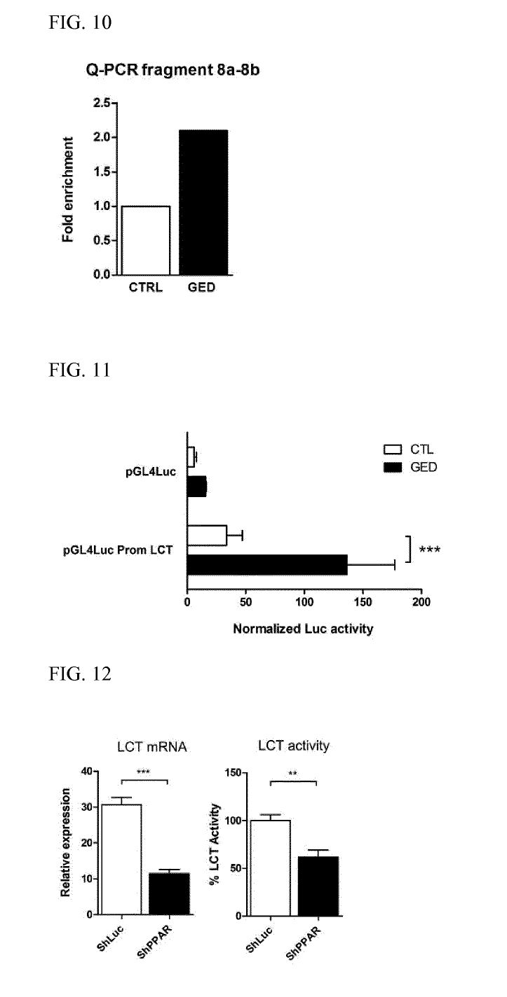

Chromatin immunoprecipitation (ChIP) was performed to determine whether these putative PPRE's are bound by PPARγ. ChIP analysis revealed that a DR2 located between −223 bp and −210 bp upstream of the LCT gene transcription start site and within the LCT gene promoter was bound by PPARγ in Caco-2 cells following stimulation for 24 hours with 1 mM GED (FIG. 10). Quantitative PCR analysis of PPARγ-bound genomic sequences demonstrated a 2-fold increase of the amount of PPARγ bound to this PPRE following 1 mM GED stimulation compared to unstimulated cells (FIG. 10).

A genomic fragment containing this PPARγ-bound DR2 sequence was cloned upstream of the luciferase gene sequence into a pGL4 vector (pGL4Luc Prom LCT construct) and tested in a reporter gene assay in Caco-2 cells. In cells transfected with the pGL4Luc Prom LCT construct, luciferase activity significantly increased in the presence of GED stimulation (GED) compared to untreated cells (CTL; FIG. 11), indicating that GED stimulated PPARγ binding to the DR2 sequence and triggered increased luciferase expression. No significant change in luciferase activity was observed in cells stably transfected with a luciferase construct lacking this DR2 sequence (pGL4Luc) following GED stimulation. These results demonstrate that the presence of this DR2 in a gene promoter facilitated PPARγ-mediated activation of downstream gene cassette expression and suggest that this DR2 response element is functional in the LCT gene promoter.

### Example 4: Lactase Gene as a PPARγ Target Gene

To further confirm the role and specificity of PPARγ in the control of LCT gene expression, a Caco-2 cell line was constructed that stably expresses a short hairpin anti-sense RNA against PPAR-γ (ShPPAR), leading to specific knockdown of PPAR-γ expression levels. In ShPPAR cells, both LCT gene transcription (FIG. 12, left) and LCT activity (FIG. 12, right) were significantly reduced by 63% and 33%, respectively, compared to Caco-2 ShLuc control cells. These results demonstrate that LCT mRNA expression and LCT activity induced by PPARγ agonist exposure was dependent upon PPARγ expression.

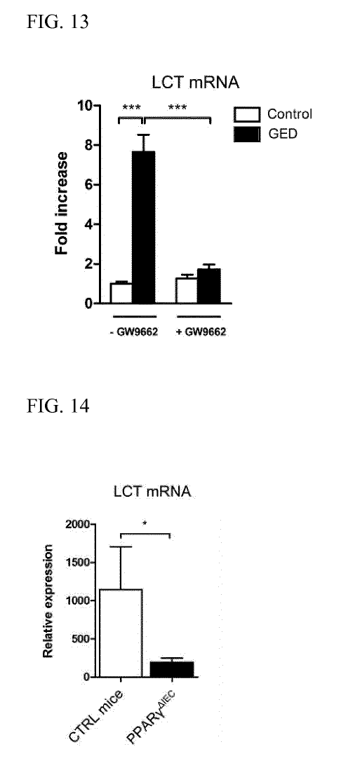

The ability of GED to induce LCT mRNA expression was analyzed in the presence of the PPAR-γ antagonist GW9662. The ability of GED to induce increased LCT mRNA expression was markedly reduced in the presence of GW9662 (FIG. 13). This result demonstrates that LCT mRNA expression and LCT activity induced by PPARγ agonist exposure was dependent upon PPARγ activity.

Expression of LCT mRNA was analyzed in the proximal small intestine of control mice (CTRL mice) and mice presenting a specific deletion of PPARγ in IEC (PPARγ^{ΔIEC} KO mice; FIG. 14). LCT mRNA expression was significantly decreased in the proximal part of the small intestine of PPARγ^{ΔIEC} KO mice compared to CTRL animals (FIG. 14). This result demonstrates that LCT expression in the proximal small intestine was dependent upon PPARγ expression.

LCT mRNA expression and PPARγ mRNA expression were significantly increased in the duodenum and jejunum of unweaned wild-type Sprague-Dawley rats compared to their weaned counterparts (FIG. 15A). Increased PPARγ and LCT mRNA expression were also significantly correlated in the duodenum and jejunum of unweaned rats (FIGS. 15B and 15C). These results demonstrate that increased PPARγ and LCT expression were significantly correlated in the proximal gut of unweaned rats.

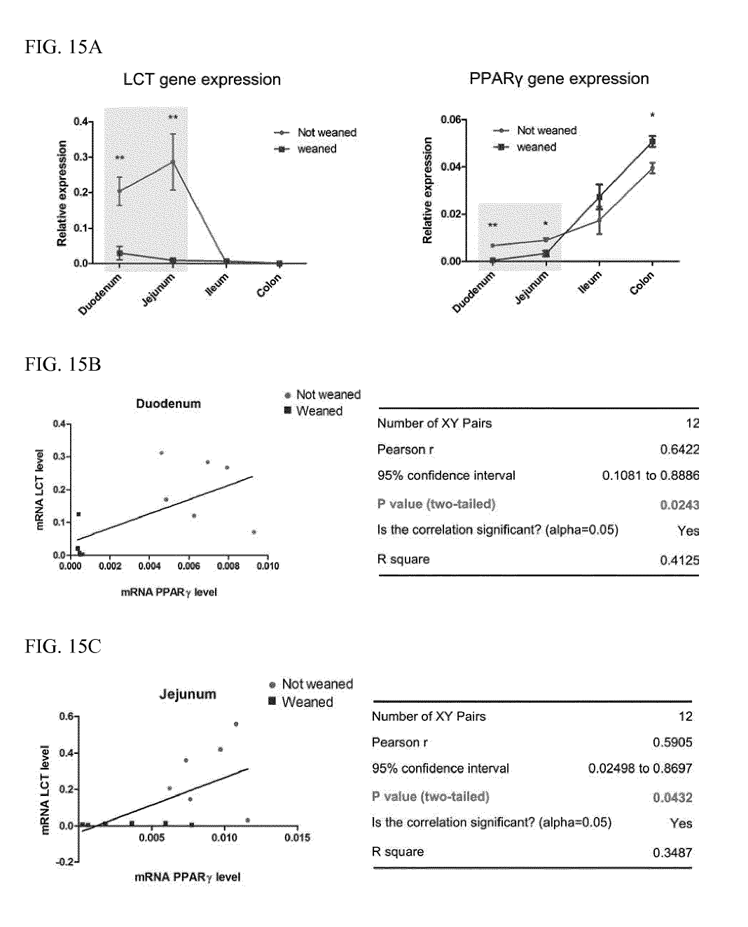

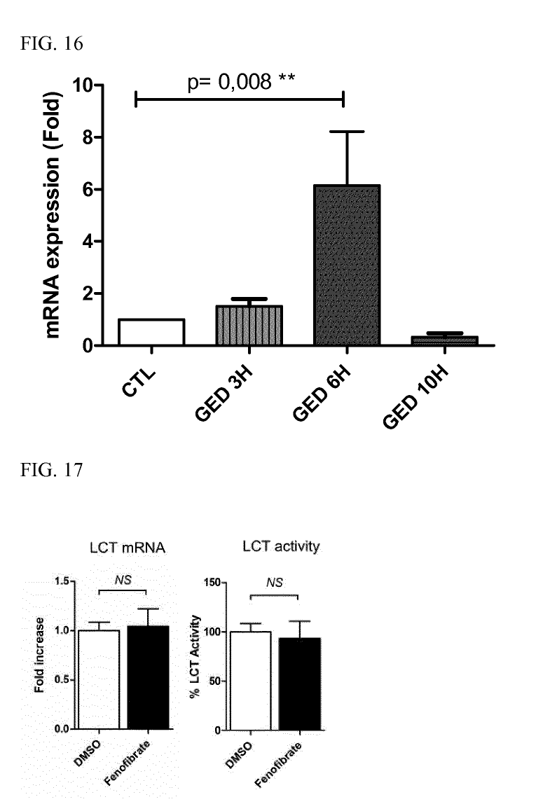

GED exposure for 6 hours also induced a significant increase in lactase expression and activity in short term cultures of human duodenal biopsies (FIG. 16 and data not shown). This result demonstrates that PPARγ agonist exposure stimulated a significant increase in LCT expression in human duodenal tissue.

Altogether, these results demonstrate that PPARγ is a key factor controlling LCT gene expression.

The potential involvement of another PPAR receptor in the control of LCT gene expression was also assessed. Fenofibrate, a specific PPARα modulator, was unable to induce and increase in LCT activity or LCT gene transcription in Caco-2 cells (FIG. 17). Moreover, PPARα expression was not modified in the ShPPARγ cell line or in IECs of PPARγ^{ΔIEC} mice (data not shown), indicating that PPARα does not play a role in regulating LCT expression. These results demonstrate that increased PPARα expression and activation did not contribute to increased LCT gene expression and activity in multiple experimental paradigms.

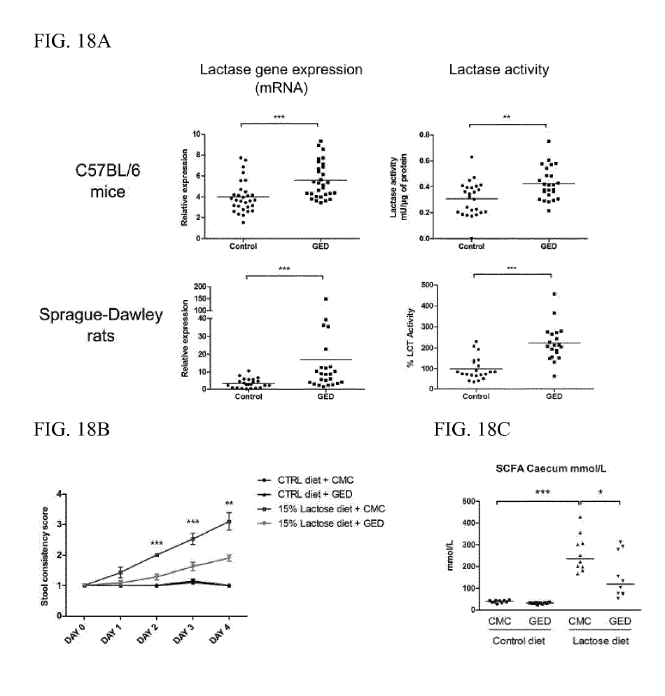

To further explore in vivo the relationship between PPARγ and LCT, the potential induction of LCT gene expression by a PPARγ modulator in rodents was assesed. Briefly, 30 mg/kg of GED was administered daily by gavage for 7 days to weaned C57BL/6 mice and Sprague-Dawley rats, and LCT activity and mRNA level were both measured in the proximal part of the small intestine. GED significantly increased LCT expression and activity in both species (FIG. 18A). These results led to testing whether GED treatment was able to improve symptoms associated with lactose intolerance. For this purpose, weaned rats that are naturally LCT non-persistent due to lack of LCT expression were fed with a lactose-enriched diet (15% or 60% of total diet weight). Compared to control animals, which received an isocaloric lactose-free diet, rats in the lactose groups lost weight and developed loose stool and diarrhoea. Stool consistency was rapidly improved by more than 40% after GED gavage in animals fed with a lactose-enriched diet (FIG. 18B). Rats were sacrificed at day 4, and rats treated with GED presented a significant decrease of caecum volume compared to untreated rats (data not shown), together with a significant decrease in concentration of short-chain fatty acids (SCFA), which corresponds to the ceacal end product of lactose fermentation (FIG. 18C). These results demonstrate that PPARγ agonist exposure stimulated increased LCT mRNA expression and LCT activity in mice and rats, and that PPARγ agonist exposure also improved symptoms associated with a lactose-enriched diet. The 40% to 50% improvement in stool consistency obtained in GED-treated rats therefore clearly suggests that modulating PPARγ activity might be clinically relevant to improving lactose tolerance in humans.

### Example 5: CLA Induces Lactase Gene Expression and Activity in an Intestinal Epithelial Cell Line

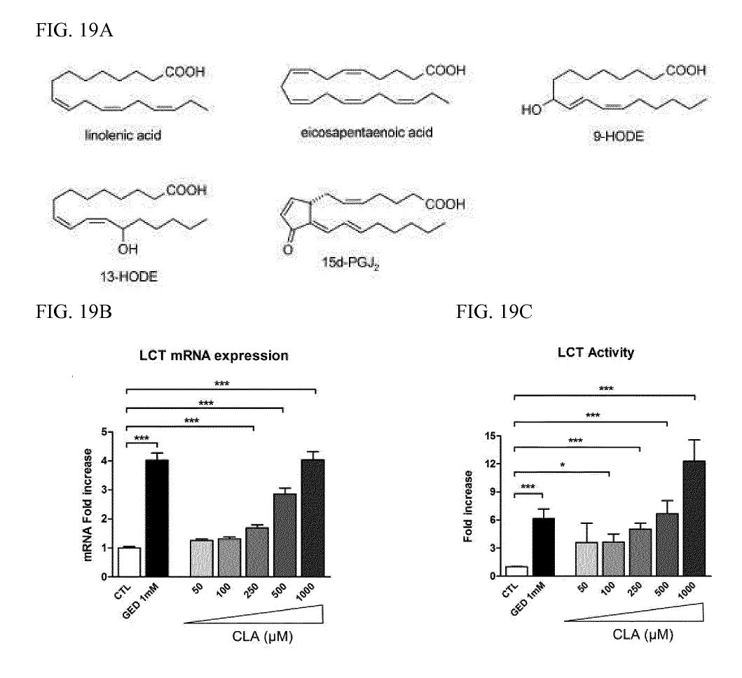

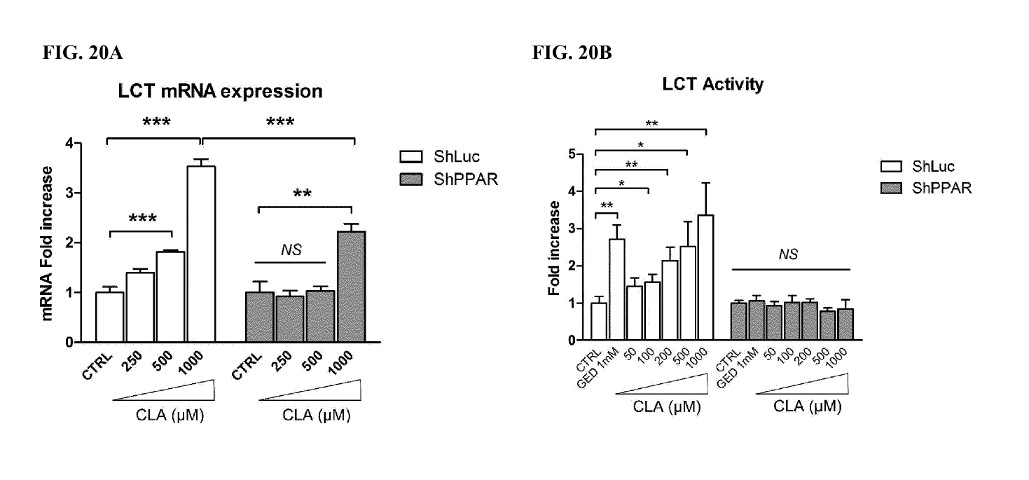

The ability of a natural PPARγ modulator, the trans-10, cis-12 conjugated linoleic acid (CLA) isomer, to induce LCT expression and activity in vitro was investigated. The structures of linolenic acid and a number of other naturally occurring PPARγ agonists are provided in FIG. 19A. CLA (1 mM) induced LCT gene expression in Caco-2 cells as efficiently as 1 mM GED (FIG. 19B; for example, compare 1 mM GED to 1 mM CLA). CLA also significantly increased LCT activity in Caco-2 cells at concentrations of 100 mM or more, and 1 mM CLA induced a 2-fold greater increase in LCT activity over control levels, compared to 1 mM GED (FIG. 19C). CLA-dependent induction of LCT expression and activity was strongly compromised in PPARγ knock-down cells (FIGS. 20A and 20B). These results demonstrate that a natural PPARγ agonist was able to induce LCT expression and activity in an intestinal epithelial cell line, and that the observed increases in LCT mRNA expression and LCT activity were dependent upon PPARγ expression. These results also strongly suggest that PPARγ modulators naturally present in food might be promising for the management of lactose intolerance.

### Example 6: CLA Induces Lactase Gene Expression and Activity In Vivo

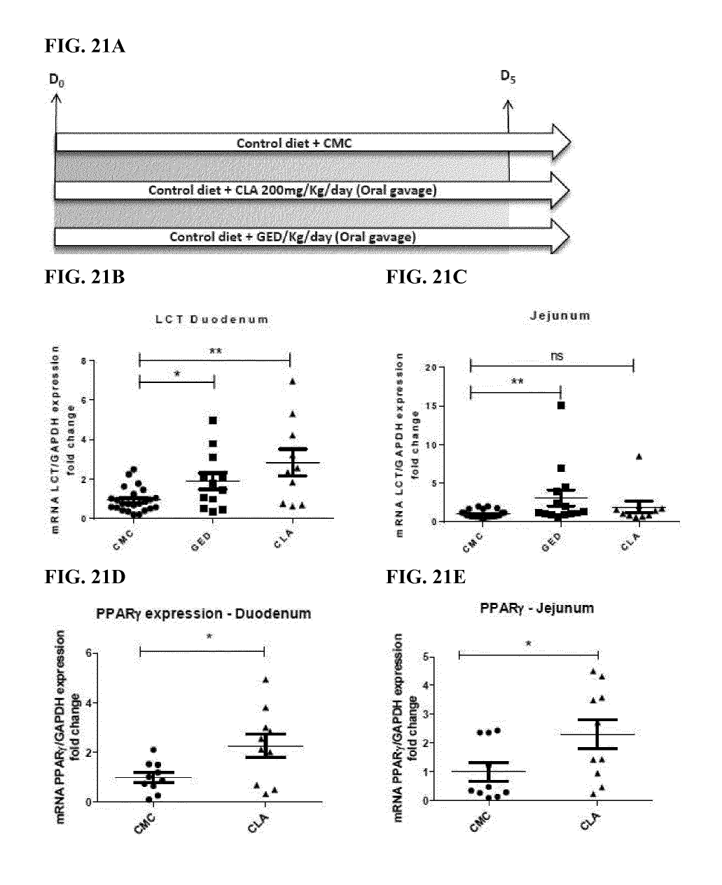

To determine whether CLA induces LCT expression and activity in vivo, Sprague Dawley rats were fed a control diet and administered either carboxymethyl cellulose (CMC; 0.5%), CLA, or GED for 5 days, after which levels of PPARγ mRNA, LCT mRNA, and LCT activity were measured. CLA was administered at 200 mg/kg/day by oral gavage, and GED was administered at 30 mg/kg/day by oral gavage (FIG. 21A).

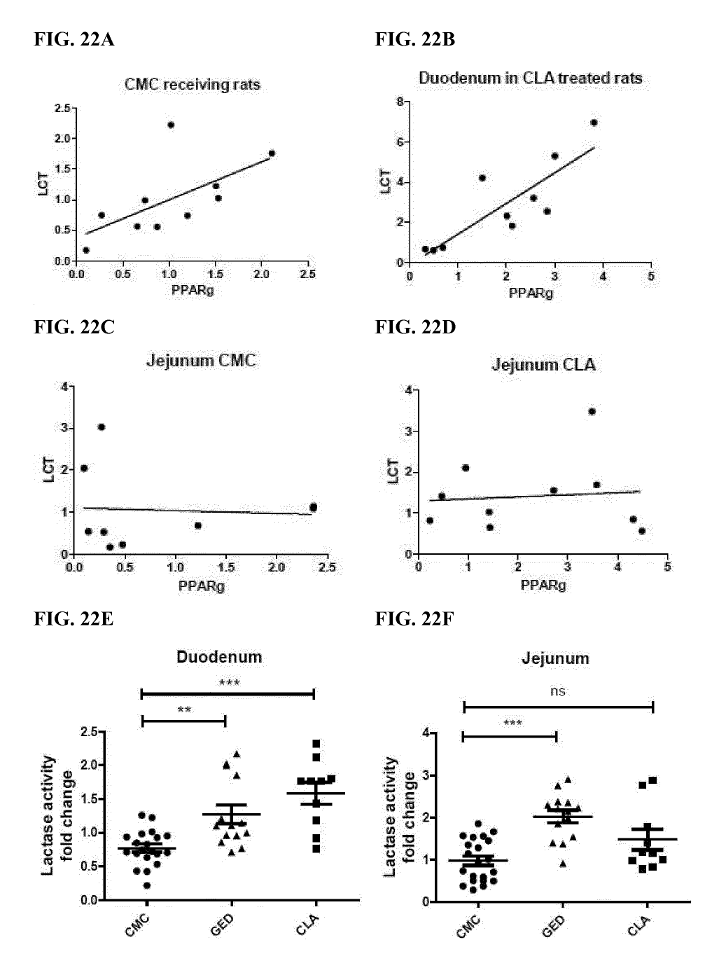

Administration of both GED and CLA resulted in a significant increase in LCT mRNA expression in duodenal tissue, relative to levels observed in animals fed a control diet supplemented with CMC (FIG. 21B). A significant increase in LCT mRNA expression in jejunal tissue, relative to levels observed in animals fed a control diet supplemented with CMC was observed following administration of GED, but not CLA (FIG. 21C). CLA administration induced a significant increase in PPARγ mRNA expression levels in both jejunum and duodenum, relative to levels observed in animals fed a control diet supplemented with CMC (FIGS. 21D and 21E). Furthermore, a significant correlation was observed between induction of LCT and PPARγ mRNA expression levels in the duodenum of animals being fed CMC or CLA (FIGS. 22A and 22B). No significant correlation was observed between induction of LCT and PPARγ mRNA expression levels in the jejunum of animals being fed CMC or CLA (FIGS. 22A and 22B). These results demonstrate that oral CLA administration for 5 days in rats resulted in a significant increase in duodenal tissue levels of LCT and PPARγ mRNA as well as a significant increase in jejunal tissue levels of PPARγ mRNA.

Levels of LCT activity resulting from oral administration of CLA were also analyzed. After 5 days, oral administration of both GED and CLA resulted in a significant increase in LCT activity in duodenal tissue as compared to LCT activity levels in animals fed a control diet supplemented with CMC (FIG. 22E). Oral administration of GED also resulted in a significant increase in LCT activity in jejunal tissue as compared to LCT activity levels in animals fed a control diet supplemented with CMC (FIG. 22F).

Unless defined otherwise, all technical and scientific terms used herein have the same meaning as commonly understood by one of ordinary skill in the art to which this invention belongs.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also include the specific quantitative value itself, unless specifically stated otherwise.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁⁻C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

As a general matter, compositions specifying a percentage are by weight unless otherwise specified. Further, if a variable is not accompanied by a definition, then the previous definition of the variable controls.

### Incorporation by Reference

All scientific articles, publications, and patent documents mentioned herein are hereby incorporated by reference in their entirety for all purposes as if each individual publication or patent was specifically and individually incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

What is claimed is:

1. A method for treating and/or ameliorating lactose intolerance or lactase deficiency in a patient in need thereof, the method comprising administering a composition comprising an isolated fatty acid to the patient.

2. A method for stimulating lactase gene expression in a patient in need thereof, the method comprising administering a composition comprising an isolated fatty acid to the patient.

3. A method for treating diarrhea, abdominal pain and/or bloating after lactose ingestion in a lactose intolerant patient in need thereof, the method comprising administering a composition comprising an isolated fatty acid.

4. The method of any one of claims 1-3, wherein the administering is before, after, or substantially concurrent with the consumption of a food that includes a dairy product.

5. The method of any one of claims 1-4, wherein the patient is also suffering from one or more of: gastroenteritis, celiac disease, Crohn's disease, and/or bacterial overgrowth.

6. The method of any one of claims 1-4, wherein the patient is undergoing radiation therapy and/or chemotherapy.

7. The method of any one of claims 1-5, wherein the administering is daily, weekly, or as needed over 3 months, 6 months, 1 year or more.

8. The method of any one of claims 1-7, wherein the fatty acid is linoleic acid, a conjugated linoleic acid, or a mixture thereof.

9. The method of claim 8, wherein the conjugated linoleic acid is selected from the group consisting of a trans-10, cis-12 conjugated linoleic acid isomer, a cis-9, trans-11 conjugated linoleic acid isomer, and mixtures thereof.

10. A method for treating and/or ameliorating lactose intolerance or lactase deficiency in a patient in need thereof, the method comprising administering to the patient a composition consisting essentially of linoleic acid, a conjugated linoleic acid, or a mixture thereof.

11. A food product comprising a therapeutically effective amount of a fatty acid to ameliorate lactose intolerance in a patient, and optionally, a dairy component.

12. The food product of claim 11, wherein the fatty acid is linoleic acid, a conjugated linoleic acid, or a mixture thereof.

13. The food product of claim 12, wherein the conjugated linoleic acid is selected from the group consisting of a trans-10, cis-12 conjugated linoleic acid isomer, a cis-9, trans-11 conjugated linoleic acid isomer, and mixtures thereof.

14. The food product of any one of claims 11-13, wherein the dairy component is whey, milk, cheese or cream.

15. A food product comprising a conjugated linoleic acid in an amount significantly greater than a naturally occurring amount of conjugated linoleic acid in the food product.

16. The food product of claim 15, wherein the amount of the conjugated linoleic acid is about 5%, about 10%, about 50%, about 100%, or more than 100% by weight greater than a naturally occurring amount of conjugated linoleic acid in the food product.

17. The food product of claim 16, wherein the conjugated linoleic acid is selected from the group consisting of a trans-10, cis-12 conjugated linoleic acid isomer, a cis-9, trans-11 conjugated linoleic acid isomer, and mixtures thereof.

18. A nutraceutical composition comprising a therapeutically effective amount of a conjugated linoleic acid, wherein the therapeutically effective amount of the conjugated linoleic acid substantially prevents, ameliorates, or treats lactose intolerance in a human patient when orally administered or consumed.

19. The nutraceutical composition of claim 18 wherein the conjugated linoleic acid is selected from the group consisting of a trans-10, cis-12 conjugated linoleic acid isomer, a cis-9, trans-11 conjugated linoleic acid isomer, and mixtures thereof.

20. A pharmaceutical formulation for oral administration of a fatty acid comprising a fatty acid, a pharmaceutically acceptable filler, and an enteric coating.

21. The pharmaceutical formulation of claim 20, wherein the fatty acid is linoleic acid, a conjugated linoleic acid, or a mixture thereof.

22. The pharmaceutical formulation of claim 20 or 21, wherein the fatty acid is a trans-10, cis-12 conjugated linoleic acid isomer, a cis-9, trans-11 conjugated linoleic acid isomer, or a mixture thereof.

23. The pharmaceutical formulation of any one of claims 20-22, further comprising a disintegrant.

24. The pharmaceutical formulation of any one of claims 20-23, further comprising a lubricant.

25. The pharmaceutical formulation of any one of claims 20-24, wherein the enteric coating is about 1% to about 10%, about 5% to about 10%, about 8% to about 10%, about 8% to about 12%, about 8% to about 15%, about 8% to about 20%, about 10% to about 12%, about 10% to about 18%, or about 15% to about 20% by weight of the pharmaceutical formulation.

26. The pharmaceutical formulation of any one of claim 20-25, wherein the enteric coating is ethylacrylate methacrylic acid.

27. The pharmaceutical formulation of any one of claim 20-26, wherein when orally administered to a patient, results in delivering the fatty acid to the duodenum.

28. The pharmaceutical formulation of any one of claim 20-27, wherein when orally administered to a patient, results in release of the fatty acid at a pH value of about 4.5, about 5, about 5.5, about 6, about 6.5, or about 7.

29. The pharmaceutical formulation of any one of claim 20-28, wherein when orally administered to a patient in need thereof, results in amelioration or treatment of lactose intolerance or lactase deficiency in the patient.

30. The pharmaceutical formulation of claim 29, wherein amelioration or treatment of lactose intolerance or lactase deficiency in the patient occurs after administering the formulation 1 time, 2 times, 3 times, 4 times, 5 times, 6 times, 7 times, 8 times, 9 times, 10 times, or more than 10 times over the course of 1 hour, 1 day, 1 week, or 1 month.
